(19) United States
(12) Patent Application Publication
(10) Pub. No.: US 2019/0274538 A1
(43) Pub. Date: Sep. 12, 2019

(54) IMAGE PROCESSING DEVICE, IMAGE PROCESSING METHOD, AND PROGRAM

(71) Applicant: CANON KABUSHIKI KAISHA, Tokyo (JP)

(72) Inventors: Hiroshi Imamura, Kawasaki-shi (JP); Hiroki Uchida, Tokyo (JP); Riuma Takahashi, Tokyo (JP)

(21) Appl. No.: 16/289,224

(22) Filed: Feb. 28, 2019

(30) Foreign Application Priority Data
Mar. 12, 2018 (JP) 2018-044559
Mar. 12, 2018 (JP) 2018-044560
Mar. 12, 2018 (JP) 2018-044563

(51) Int. Cl. A61B 3/00; A61B 3/12; A61B 3/10; G06T 7/00

(52) U.S. Cl. CPC A61B 3/0058; A61B 3/1241; A61B 3/102; G06T 2207/30104; G06T 2207/10101; G06T 2207/20221; G06T 2207/30041; G06T 7/0016

(57) ABSTRACT

An image processing device is provided and includes an acquiring unit configured to acquire combined images associated with examination dates, each of combined images being obtained by using motion contrast images of a portion of an eye portion, and a display control unit configured to cause a display unit to display the combined images in time series and to cause the display unit to display a plurality of pieces of information regarding the examination dates in time series, the plurality of pieces of information being obtained by using the combined images.

### BACKGROUND

### Field

The present disclosure relates to an image processing device, an image processing method, and a program.

### Description of the Related Art

OCT Angiography (hereinafter referred to as OCTA) that non-invasively extracts a fundus oculi blood vessel by using an optical coherence tomography (OCT) is known. In OCTA, the same position is scanned by measurement light a plurality of times, and a plurality of OCT tomographic images are acquired. Motion contrast data obtained by interaction of displacement of red blood cells and the measurement light based on the plurality of OCT tomographic images is imaged as an OCTA image.

Japanese Patent Laid-Open No. 2017-77414 discloses a technique of juxtaposing and displaying, in time series, blood vessel analysis maps calculated for each of a plurality of motion contrast data whose acquisition periods (examination dates) are different.

### SUMMARY

To achieve an object of the present disclosure, an image processing device includes an acquiring unit configured to acquire combined images associated with examination dates, each of combined images being obtained by using motion contrast images of a portion of an eye portion, and a display control unit configured to cause a display unit to display the combined images in time series and to cause the display unit to display a plurality of pieces of information regarding the examination dates in time series, the plurality of pieces of information being obtained by using the combined images.

Further features will become apparent from the following description of exemplary embodiments (with reference to the attached drawings).

### BRIEF DESCRIPTION OF THE DRAWINGS

FIG. 1 is a block diagram showing an example of an image processing device according to a first embodiment.

FIGS. 2A and 2B are diagrams for explaining an example of an image processing system according to an embodiment and an example of a measurement optical system included in a tomographic image capturing device that configures the image processing system.

FIG. 3 is a flowchart showing an example of processing that can be performed by the image processing system according to the first embodiment.

FIG. 4 is a diagram for explaining an example of a scanning method of OCTA image capturing in the embodiment.

FIGS. 5A to 5C are diagrams for explaining an example of processing performed in S307 of the first embodiment.

FIGS. 6A and 6B are diagram for explaining an example of processing performed in S308 of the first embodiment.

FIGS. 7A and 7B are diagrams for explaining an example of a selection screen of a reference examination and an example of an image capturing screen, which are displayed on the display unit in the first embodiment.

FIGS. 8A to 8E are diagrams for explaining an example of an image processing content in S304 and an example of a report screen displayed on the display unit in S305 in the first embodiment.

FIGS. 9A to 9B are diagrams for explaining an example of a measurement operation screen displayed on the display unit and an example of a measurement report screen displayed in S308 in the first embodiment.

FIGS. 10A to 10F are diagrams for explaining an example of an operation procedure when a user modifies a specified blood vessel area and an example of an image processing content to be performed in the first embodiment.

FIG. 11 is a diagram for explaining an example of a temporal change measurement report screen displayed on the display unit in S311 in the first embodiment.

FIG. 12 is a diagram for explaining a measurement report screen on which a warning message is displayed in the first embodiment.

### DESCRIPTION OF THE EMBODIMENTS

### First Embodiment

It is expected that a change of a fundus oculi blood vessel can be quantitatively grasped by evaluating an eye blood vessel in time series. However, an OCTA image varies for each examination according to signal intensity or image quality variation of an OCT tomographic image for each examination. Specifically, even when no temporal change occurs in the eye blood vessel, there is a case where a temporal change appears in the fundus oculi blood vessel in the OCTA image. That is, there is a case where the temporal change of the fundus oculi blood vessel cannot be appropriately evaluated. An object of the present embodiment is to support appropriate evaluation of temporal change regarding a fundus oculi blood vessel.

Specifically, an image processing device according to the present embodiment performs blood vessel area identification processing and blood vessel density measurement processing by using front motion contrast images of retinal surface and retinal deep layer generated from an OCTA superimposed image (a combined image of a plurality of OCTA images) acquired from the same examinee eye on different dates in substantially the same image capturing condition. A case will be described where combined images and measurement values obtained by the identification processing and measurement processing are juxtaposed and displayed in time series in a plurality of depth ranges. Here, substantially the same image capturing condition is, for example, a condition of follow-up image capturing intended for follow-up observation. In the present disclosure, the follow-up image capturing intended for follow-up observation and image superimposition processing are not essential.

Hereinafter, an image processing system including the image processing device according to the first embodiment will be described with reference to the drawings.

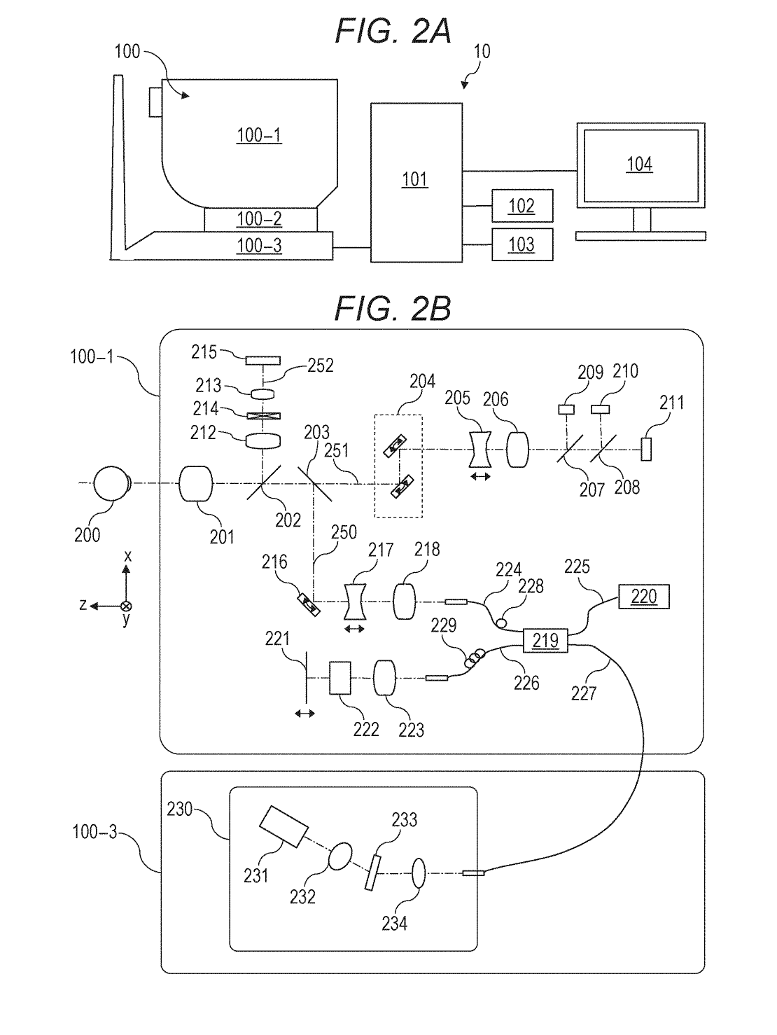

FIGS. 2A and 2B are diagrams showing a configuration of an image processing system 10 including an image processing device 101 according to the present embodiment. As shown in FIGS. 2A and 2B, the image processing system 10 is configured by connecting the image processing device 101 to a tomographic image capturing device 100 (also referred to as OCT), an external storage unit 102, an input unit 103, and a display unit 104 through an interface. The input unit 103 may be a touch panel. When the input unit 103 is a touch panel, the input unit 103 is integrated with the display unit 104. The image processing device 101 may be included inside the tomographic image capturing device 100.

The tomographic image capturing device 100 is a device that captures a tomographic image of an eye portion. In the present embodiment, an SD-OCT (Spectral Domain OCT) is used as the tomographic image capturing device 100. The tomographic image capturing device 100 is not limited to the SD-OCT, but the tomographic image capturing device 100 may be configured by using an SS-OCT (Swept Source OCT).

In FIG. 2A, a measurement optical system 100-1 is an optical system for acquiring an anterior eye portion image, an SLO (Scanning Laser Ophthalmoscopy) fundus image of an examinee eye, and a tomographic image. The optical system for acquiring a fundus image is not limited to an SLO optical system, but may be a fundus camera. A stage portion 100-2 makes the measurement optical system 100-1 movable back and forth and right and left. A base portion 100-3 incorporates a spectrometer described later.

The image processing device 101 is a computer that performs control of the stage portion 100-2, control of an alignment operation, reconstruction of a tomographic image, and the like. The external storage unit 102 stores a program for capturing a tomographic image, a patient information, captured image data, image data and measurement data of past examinations, and the like.

The input unit 103 issues an instruction to the computer and is specifically composed of a keyboard and a mouse. The display unit 104 is composed of, for example, a monitor.

(Configuration of Tomographic Image Capturing Device)

Configurations of the measurement optical system and the spectrometer in the tomographic image capturing device 100 of the present embodiment will be described with reference to FIG. 2B.

First, the inside of the measurement optical system 100-1 will be described. An objective lens 201 is installed facing an examinee eye 200, and a first dichroic mirror 202 and a second dichroic mirror 203 are arranged on an optical axis of the objective lens 201. An optical path is branched by these dichroic mirrors for each wavelength band into an optical path 250 for an OCT optical system, an optical path 251 for an SLO optical system and a fixation lamp, and an optical path 252 for observing an anterior eye.

The optical path 251 for the SLO optical system and the fixation lamp has an SLO scanning unit 204, lenses 205 and 206, a mirror 207, a third dichroic mirror 208, an APD (Avalanche Photodiode) 209, an SLO light source 210, and a fixation lamp 211.

The mirror 207 is a prism vapor-deposited with a perforated mirror and/or a hollow mirror. The mirror 207 separates illumination light from the SLO light source 210 and return light from the examinee eye from each other. The third dichroic mirror 208 separates the optical path for each wavelength band into an optical path of the SLO light source 210 and an optical path of the fixation lamp 211.

The SLO scanning unit 204 scans light emitted from the SLO light source 210 on the examinee eye 200. The SLO scanning unit 204 includes an X scanner that scans in an X direction and a Y scanner that scans in a Y direction. In the present embodiment, the X scanner has to perform high-speed scanning, so that the X scanner is a polygonal mirror. The Y scanner is composed of a Galvano mirror. The configurations of the scanners are not limited to the examples described above. For example, the X scanner may also be composed of a Galvano mirror.

The lens 205 is driven by a motor not shown in the drawings for focusing of the SLO optical system and the fixation lamp 211. The SLO light source 210 generates light having a wavelength of, for example, about 780 nm. In the present Specification, numerical values such as a wavelength and the like are examples, and may be changed to other numerical values. The APD 209 detects the return light from the examinee eye. The fixation lamp 211 generates visible light and urges an examinee to fix his or her visual line.

The light emitted from the SLO light source 210 is reflected by the third dichroic mirror 208, passes through the mirror 207, passes through the lenses 206 and 205, and is scanned on the examinee eye 200 by the SLO scanning unit 204. The return light from the examinee eye 200 returns through the same path as that of the illumination light, and thereafter is reflected by the mirror 207 and guided to the APD 209, so that an SLO fundus image is obtained.

The light emitted from the fixation lamp 211 transmits through the third dichroic mirror 208 and the mirror 207, passes through the lenses 206 and 205, forms a predetermined shape in an arbitrary position on the examinee eye 200 by the SLO scanning unit 204, and urges an examinee to fix his or her visual line.

In the optical path 252 for observing the anterior eye, lenses 212 and 213, a split prism 214, and a CCD 215 that detects infrared light for observing the anterior eye portion are arranged. The CCD 215 has a sensibility in a wavelength of irradiation light for observing the anterior eye portion not shown in the drawings, specifically a wavelength around 970 nm. The split prism 214 is arranged in a position conjugate to a pupil of the examinee eye 200 and can detect a distance to the examinee eye 200 in Z axis direction (optical axis direction) of the measurement optical system 100-1 as a split image of the anterior eye portion.

The optical path 250 of the OCT optical system constitutes the OCT optical system as described above and is to capture a tomographic image of the examinee eye 200. More specifically, the optical path 250 is to obtain an interfering signal for forming a tomographic image. An XY scanner 216 is to scan light on the examinee eye 200. The XY scanner 216 is shown as one mirror in FIG. 2B. However, the scanner 216 is actually a Galvano mirror that performs scan in X and Y axis directions.

A lens 217 of lenses 217 and 218 is driven by a motor (not shown in the drawings) to focus light from an OCT light source 220, which is emitted from a fiber 224 connected to an optical coupler 219, to the examinee eye 200. By this focusing, the return light from the examinee eye 200 is formed into an image in a spot shape on a leading edge of the fiber 224 and inputted into the leading edge of the fiber 224 at the same time. Next, an optical path from the OCT light source 220, a reference optical system, and a configuration of a spectrometer will be described. Reference numeral 220 denotes the OCT light source, reference numeral 221 denotes a reference mirror, reference numeral 222 denotes a dispersion compensation glass, reference numeral 223 denotes a lens, reference numeral 219 denotes the optical coupler, reference numerals 224 to 227 denote optical fibers in a single mode which are connected to the optical coupler, and reference numeral 230 denotes the spectrometer.

A Michelson interferometer is configured by the components described above. The light emitted from the OCT light source 220 passes through the optical fiber 225 and is divided into measurement light on the optical fiber 224 side and reference light on the optical fiber 226 side through the optical coupler 219. The measurement light is irradiated to the examinee eye 200, which is an object to be observed, through the optical path of the OCT optical system described above and reaches the optical coupler 219 through the same optical path by reflection and scattering from the examinee eye 200.

On the other hand, the reference light reaches the reference mirror 221 through the optical fiber 226, the lens 223, and the dispersion compensation glass 222 inserted in order to balance wavelength dispersion of the measurement light and the reference light, and is reflected by the reference mirror 221. Then the reference light returns through the same optical path and reaches the optical coupler 219.

The measurement light and the reference light are multiplexed into interference light by the optical coupler 219.

Here, when an optical path length of the measurement light and an optical path length of the reference light are substantially the same, interference occurs. The reference mirror 221 is held in an adjustable manner in an optical axis direction by a motor and a driving mechanism, which are not shown in the drawings, and the optical path length of the reference light can be adjusted to the optical path length of the measurement light. The interference light is guided to the spectrometer 230 through the optical fiber 227.

Polarization adjusting units 228 and 229 are provided in the optical fibers 224 and 226, respectively, and adjust polarization. These polarization adjusting units have some portions where an optical fiber is drawn in a loop shape. Polarization states of the measurement light and the reference light can be adjusted, respectively, and matched with each other by applying a twist to the fiber by rotating the loop-shaped portion around the longitudinal direction of the fiber.

The spectrometer 230 is composed of lenses 232 and 234, a diffractive grating 233, and a line sensor 231. The interference light emitted from the optical fiber 227 becomes parallel light through the lens 234 and then is dispersed by the diffractive grating 233 and formed into an image on the line sensor 231 by the lens 232.

Next, the periphery of the OCT light source 220 will be described. The OCT light source 220 is an SLD (Super Luminescent Diode) which is a typical low-coherence light source. The central wavelength is 855 nm, and the wavelength bandwidth is about 100 nm. Here, the bandwidth affects resolution of the obtained tomographic image in the optical axis direction, so that the bandwidth is an important parameter.

Here, the SLD is selected as the type of the light source. However, the light source only has to emit low-coherence light, and ASE (Amplified Spontaneous Emission) or the like can be used. Near-infrared light is suitable as the central wavelength when considering that an eye is measured. Further, the central wavelength affects resolution in the horizontal direction of the obtained tomographic image, so that it is desirable that the central wavelength is as short as possible. From the above reasons, the central wavelength is determined to be 855 nm.

In the present embodiment, the Michelson interferometer is used as an interferometer. However, a Mach-Zehnder interferometer may also be used. It is desirable to use the Mach-Zehnder interferometer when a difference of light quantity between the measurement light and the reference light is large and use the Michelson interferometer when the difference of light quantity is relatively small.

(Configuration of Image Processing Device)

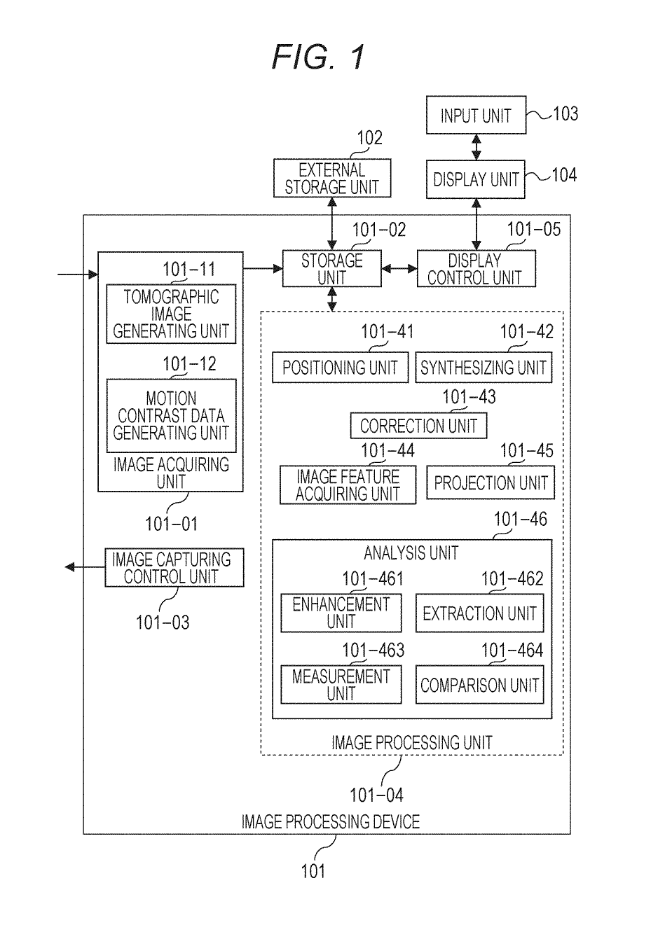

A configuration of the image processing device 101 of the present embodiment will be described with reference to FIG. 1.

The image processing device 101 is a personal computer (PC) connected to the tomographic image capturing device 100. The image processing device 101 includes an image acquiring unit 101-01, a storage unit 101-02, an image capturing control unit 101-03, an image processing unit 101-04, and a display control unit 101-05. Functions of the image processing device 101 are realized when an arithmetic processing device CPU executes a software module that realizes the image acquiring unit 101-01, the image capturing control unit 101-03, the image processing unit 101-04, and the display control unit 101-05. For example, when a processor such as a CPU included in the image processing device 101 executes a program stored in the storage unit 101-02, the processor functions as the image acquiring unit 101-01., the image capturing control unit 101-03, the image processing unit 101-04, and the display control unit 101-05. The present disclosure is not limited to this. For example, the image processing unit 101-04 may be realized by dedicated hardware such as an ASIC, and the display control unit 101-05 may be realized by using a dedicated processor such as a GPU different from the CPU. The tomographic image capturing device 100 and the image processing device 101 may be connected through a network.

The image acquiring unit 101-01 acquires signal data of an SLO fundus image and a tomographic image captured by the tomographic image capturing device 100. The image acquiring unit 101-01 has a tomographic image generating unit 101-11 and a motion contrast data generating unit 101-12. The tomographic image generating unit 101-11 acquires signal data (interfering signal) of the tomographic image captured by the tomographic image capturing device 100, generates a tomographic image by signal processing, and stores the generated tomographic image into the storage unit 101-02. The motion contrast data generating unit 101-12 generates motion contrast data from a plurality of tomographic images (tomographic data).

The image capturing control unit 101-03 performs image capturing control on the tomographic image capturing device 100. The image capturing control includes issuing an instruction regarding setting of an image capturing parameter and an instruction regarding start or end of image capturing to the tomographic image capturing device 100.

The image processing unit 101-04 has a positioning unit 101-41, a synthesizing unit 101-42, a correction unit 101-43, an image feature acquiring unit 101-44, a projection unit 101-45, and an analysis unit 101-46. The image acquiring unit 101-01 described above and the synthesizing unit 101-42 are an example of an acquiring unit. At this time, the synthesizing unit 101-42 generates a synthesized motion contrast image by synthesizing a plurality of motion contrast data generated by the motion contrast data generating unit 101-12 based on a positioning parameter obtained by the positioning unit 101-41. Further, the synthesizing unit 101-42 generates the synthesized motion contrast image for each of a plurality of examination dates. The synthesizing unit 101-42 corresponds to an example of the acquiring unit that acquires a combined image of a plurality of motion contrast images regarding each of a plurality of examination dates. The synthesizing unit 101-42 may generate a synthesized motion contrast image by synthesizing (additionally averaging) a plurality of three-dimensional motion contrast images or may generate a synthesized motion contrast image by synthesizing a plurality of two-dimensional motion contrast images. The plurality of tomographic images to be a source of the motion contrast image in the present embodiment are images captured by scanning light in the same main scanning direction.

The correction unit 101-43 performs processing for two-dimensionally or three-dimensionally suppressing projection artifact generated in the motion contrast image (the projection artifact will be described in S304). The image feature acquiring unit 101-44 acquires a layer boundary of retina and choroid, fovea, and a center position of optic disk from the tomographic image. The projection unit 101-45 projects the motion contrast image in a depth range based on a position of the layer boundary acquired by the image feature acquiring unit 101-44 and generates a front motion contrast image (En Face image of OCTA). The analysis unit 101-46 has an enhancement unit 101-461, an extraction unit 101-462, a measurement unit 101-463, and a comparison unit 101-464, and performs extraction processing and measurement processing of a blood vessel area from the front motion contrast image. That is, the analysis unit 101-46 performs extraction and the like of the blood vessel area from a two-dimensional motion contrast image. Here, the analysis unit 101-46 is an example of an analysis unit that performs an analysis on at least one area selected from a first area and a second area that includes at least an area smaller than the first area in a motion contrast image of an eye portion. The second area is an example of a sector area. The first area is an example of an area larger than the sector area (for example, the whole image).

The analysis unit 101-46 may perform an analysis on at least a partial area of a motion contrast image of an eye portion. The enhancement unit 101-461 generates a blood vessel enhanced image by performing blood vessel enhancement processing on the front motion contrast image. The extraction unit 101-462 extracts the blood vessel area based on the blood vessel enhanced image. The measurement unit 101-463 calculates measurement values such as blood vessel density by using the extracted blood vessel area and blood vessel center line data acquired by thinning the blood vessel area. The comparison unit 101-464 generates temporal comparison data by reading synthesized motion contrast images of the same examinee eye acquired on different examination dates and accompanying measurement data from the storage unit 101-02 or the external storage unit 102. The comparison unit 101-464 corresponds to an example of the acquiring unit that acquires a combined image of a plurality of motion contrast images regarding each of a plurality of examination dates. It is preferable that the display control unit 101-05 uses information indicating a type of analysis selected for one of the first and the second areas according to an instruction from an operator and thereby causes the display unit 104 to display an image showing a result where the one of the first and the second areas is analyzed.

Here, the types of the selected analysis are, for example, a blood vessel density regarding the area of the blood vessel area (Vessel Area Density; VAD) and a blood vessel density regarding a blood vessel length (Vessel Length Density; VLD), and the like. An image showing an analyzed result is, for example, a two-dimensional image showing a result obtained by analyzing at least a partial area of a motion contrast front image. The two-dimensional image showing the analyzed result is, for example, a VAD map, a VLD map, a VAD sector map, a VLD sector map, and an image where these analysis maps are superimposed on the motion contrast front image. Further, the two-dimensional image showing the analyzed result may be an image where a plurality of analysis maps of the same type are superimposed and an image where a plurality of analysis maps of the same type are superimposed on the motion contrast front image. For example, there are a two-dimensional image where the VAD sector map is superimposed on the VAD map, a two-dimensional image where the VAD sector map and the V AD map are superimposed on the motion contrast image, a two-dimensional image where the VLD sector map is superimposed on the VLD map, and a two-dimensional image where the VLD sector map and the VLD map are superimposed on the motion contrast image. The timing when the analysis unit 101-46 performs an analysis may be a timing when the type of analysis is selected according to an instruction from the operator. Alternatively, before the type of analysis is selected, an analysis corresponding to the type of supposed analysis may be completed in advance.

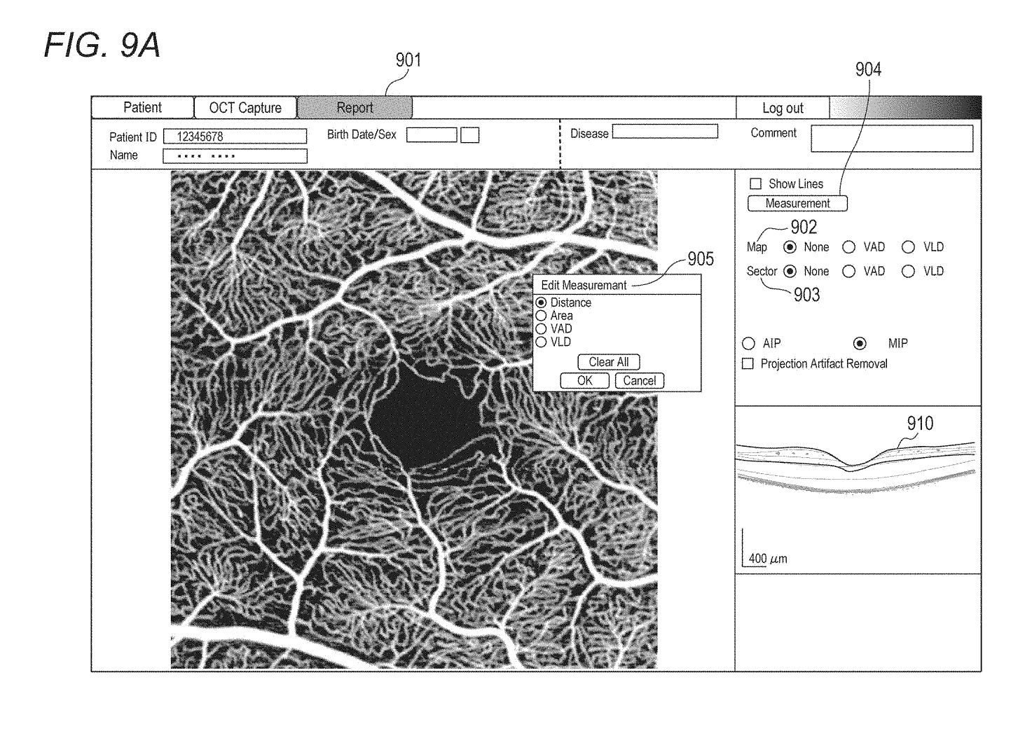

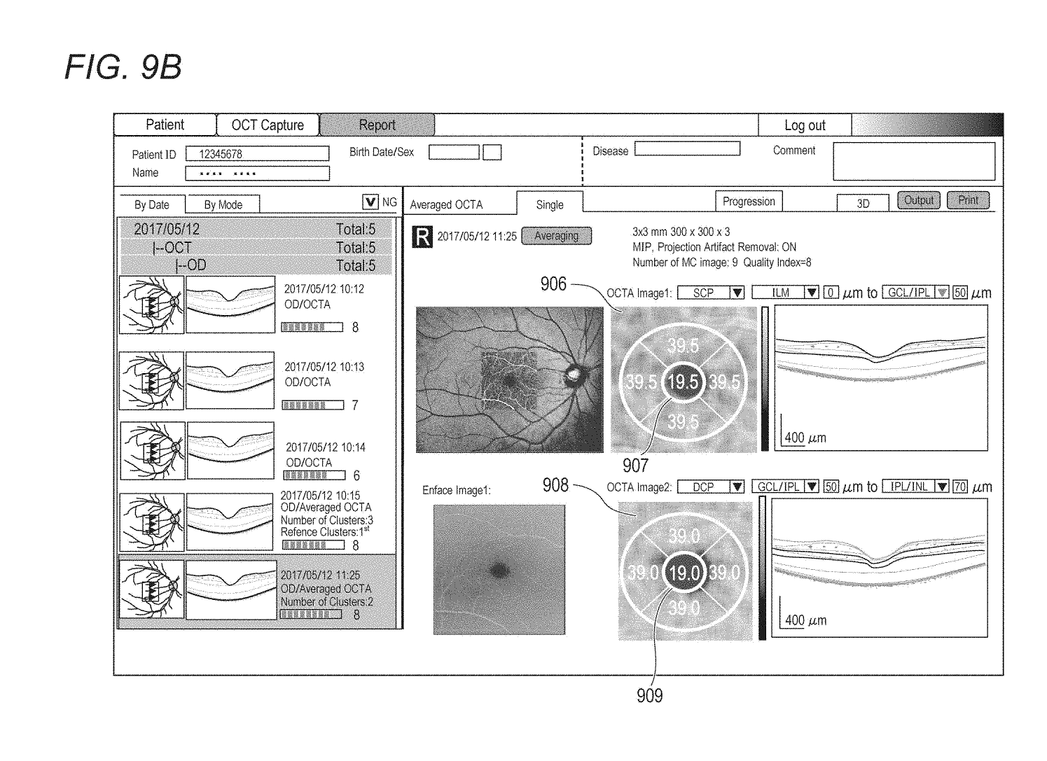

Here, a case is considered where the type of analysis selected for the other area according to an instruction from the operator after selection for one area is different from the type of analysis selected for the one area. At this time, it is preferable that in a display area of an image showing a result obtained by analyzing the one area, the display control unit 101-05 performs control to change the display of the image showing the result obtained by analyzing the one area to a display of an image showing a result obtained by analyzing the one area and the other area by using information indicating the type of analysis selected for the other area. Thereby, when an image showing a result of analysis performed on a plurality of analysis areas on the motion contrast image is displayed, it is possible to configure so that selection of types corresponding to each other can be easily performed as types of analysis on a plurality of analysis areas. For example, after the VLD map is selected in a Map button group 902 on the right side of FIG. 9A, when the VAD sector is selected in a Sector button group 903, it is preferable to perform control to change a display of a two-dimensional image where the VLD map is superimposed on the motion contrast front image to a display of a two-dimensional image where the VAD sector map and the VAD map are superimposed on the motion contrast front image. Thereby, a display where the types of analysis are different from each other such as a two-dimensional image where, for example, the VAD sector map and the VLD map are superimposed on the motion contrast image does not appear. In other words, for example, a plurality of analysis maps to be superimposed are reliably selected as the same type of analyses, so that it is possible to easily check an analysis result. At this time, of course, it is preferable to perform control to change a display of information indicating the type of analysis. Specifically, when the type of analysis selected for the other area according to an instruction from the operator after selection for one area is different from the type of analysis selected for the one area, it is preferable that the display control unit 101-05 performs another control to change a display of information indicating the type of analysis selected for the one area to a display of information indicating the type of analysis selected for the other area. Regarding the display of information indicating the type of selected analysis, anything may be displayed as long as the type of selected analysis is displayed on the display unit 104 so that the type of selected analysis can be identified. As an example, there are the Map button group 902 and the Sector button group 903 on the right side of FIG. 9A. In the follow-up image capturing intended for follow-up observation, it is preferable that the above control performed on one image of a plurality of motion contrast images is also performed on the other images in a display area where the plurality of motion contrast images corresponding to a plurality of examination dates are displayed in a time-sequential arrangement. Further, it is preferable that the information indicating the type of analysis selected for one image of a plurality of motion contrast images corresponding to a plurality of examination dates is applied to the other images. Thereby, it is possible to improve convenience in the follow-up image capturing intended for follow-up observation. The sector area is preferred to be divided into a plurality of areas, and in each area, it is preferable to display a value showing an analysis result of the area (for example, an average value of the area) in a state where a unit of the type of the analysis can be identified. Here, when “None” is selected in the Map button group 902 and/or the Sector button group 903 according to an instruction from the operator, the type of analysis is preferred to be a non-selecting state. At this time, it is preferable that the image showing the analyzed result becomes a state of non-display in a display area and the motion contrast image is displayed in the display area. When the type of analysis selected for one area is changed to non-selecting according to an instruction from the operator, the type of analysis selected for the other area is preferred to be unchanged.

Further, a case is considered where an analysis is performed on the first area in the motion contrast image of the eye portion by using information indicating the type of analysis selected according to an instruction from the operator. At this time, when display of an image showing a result obtained by analyzing the second area in the motion contrast image is selected according to an instruction from the operator, the display control unit 101-05 may cause the display unit 104 to display the image showing the result obtained by analyzing the second area by using information indicating the type of selected analysis in a state where the image is superimposed on an image showing a result obtained by analyzing the first area. Thereby, for example, when VAD is selected as the type of analysis and “On” is selected as a display of the sector area, it is possible to display a two-dimensional image where the VAD sector map is superimposed on the VAD map in the display area. Therefore, for example, a plurality of analysis maps to be superimposed are reliably selected as the same type of analyses, so that it is possible to easily check an analysis result.

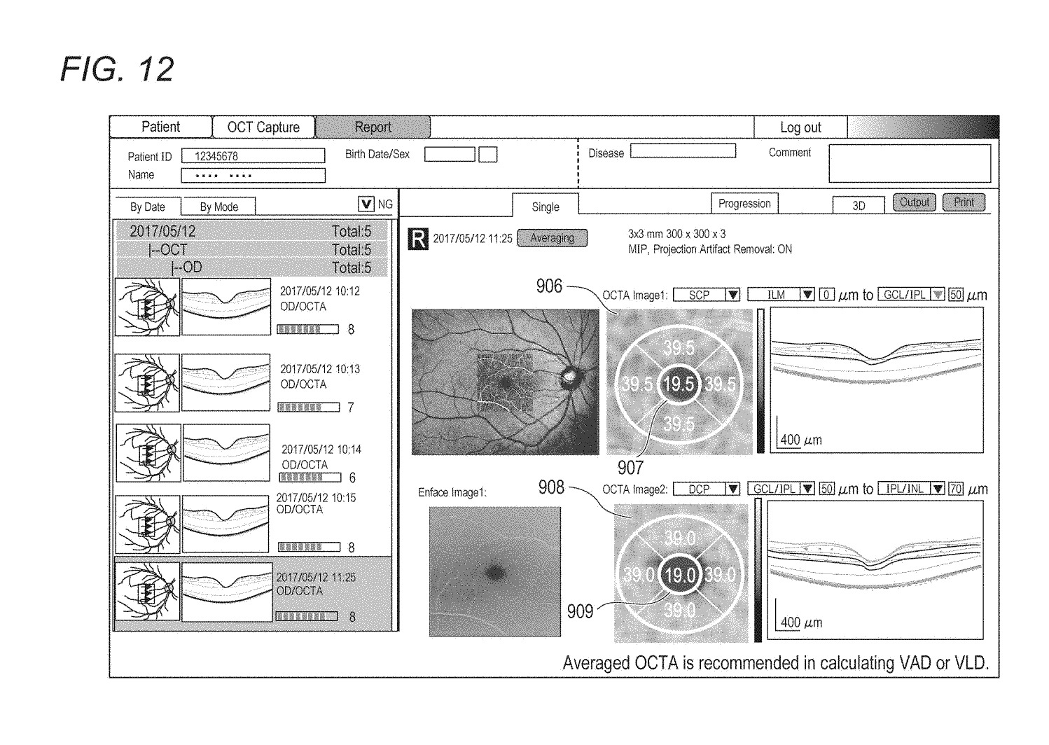

Further, the display control unit 101-05 may be an example of a reporting unit which, when an image showing an analysis result displayed on the display unit 104 according to an instruction from the operator is an image obtained in a state where at least two conditions of a plurality of conditions suitable for analysis are not satisfied, reports information regarding at least two conditions according to an order of priorities of the plurality of conditions. Thereby, even in a case where at least two conditions of a plurality of conditions suitable for analysis are not satisfied, the operator can easily cope with the case so as to obtain a more accurate analysis result. Here, it is preferable that the plurality of conditions suitable for analysis include a condition where the motion contrast image is, for example, an image obtained by synthesizing a plurality of three-dimensional motion contrast images obtained by performing control so that the measurement light scans the same position of the eye portion, as a condition whose priority is higher than those of the other conditions. Thereby, it is possible to advise the operator to check, for example, an analysis result using a high quality image. It is preferable that the display control unit 101-05 causes the display unit 104 to display information regarding at least two conditions. At this time, it is preferable that the display control unit 101-05 causes the display unit 104 to display an image showing an analysis result using information indicating the type of analysis selected according to an instruction from the operator juxtaposed with the information regarding at least two conditions. Further, it is preferable that there is a warning message regarding a higher priority condition of at least two conditions. For example, as shown in lower right part in FIG. 12, a warning message “Averaged OCTA is recommended in calculating VAD or VLD.” may be displayed in an edge or the like of a display area where an image showing an analysis result is displayed. The warning message described above may be displayed in an edge or the like of a display area, where an image showing an analysis result is displayed, in a state where the warning message is superimposed on the image showing the analysis result. Thereby, for example, while an image showing an analysis result which the operator most wants to check is displayed in a display area of the display unit 104, it is possible to advise the operator of a condition suitable for analysis by effectively using a remaining space. Of course, the reporting unit may report warning messages regarding at least two conditions, respectively, in a priority order, as information regarding at least two conditions.

The external storage unit 102 holds information of the examinee eye (name, age, sex, and the like of the patient), captured images (tomographic image, SLO image, and OCTA image), a combined image, image capturing parameters, positional data of blood vessel area and blood vessel center line, measurement values, and parameters set by the operator in association with each other. The input unit 103 is, for example, a mouse, a keyboard, a touch operation screen, and the like. The operator issues an instruction to the image processing device 101 and the tomographic image capturing device 100 through the input unit 103.

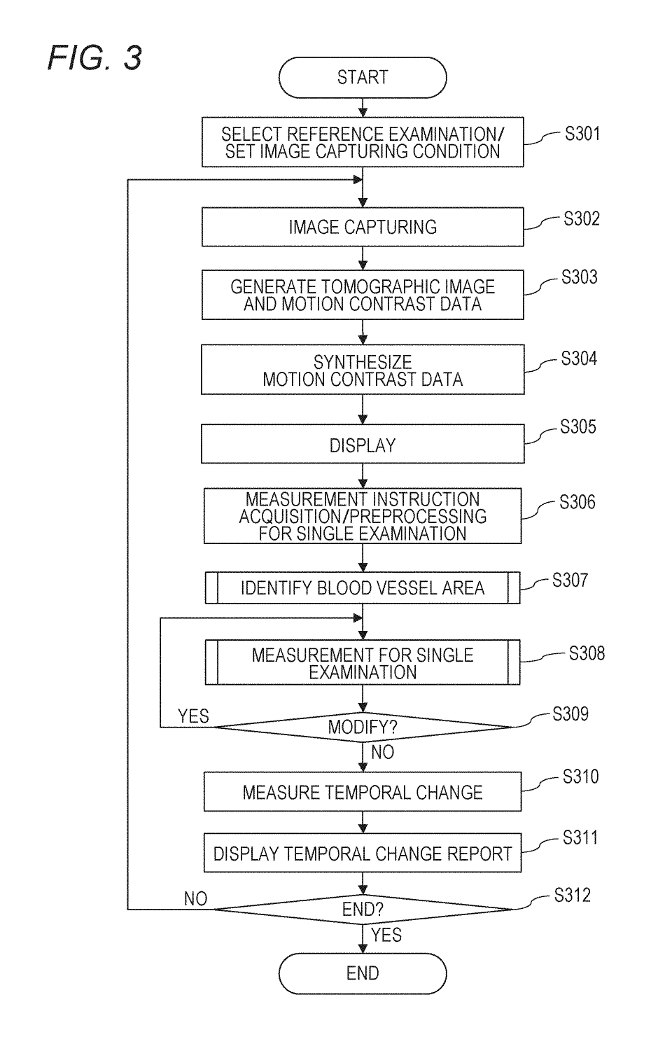

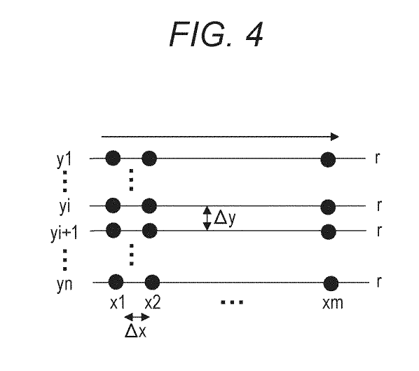

Next, a processing procedure of the image processing device 101 of the present embodiment will be described with reference to FIG. 3. FIG. 3 is a flowchart showing a flow of operation processing of the entire image processing system in the present embodiment.

<Step 301>

The operator selects a reference examination regarding a examinee eye whose past examination data is stored. The image processing device 101 sets an image capturing condition of OCTA image capturing so that the image capturing condition is the same as that of the selected reference examination.

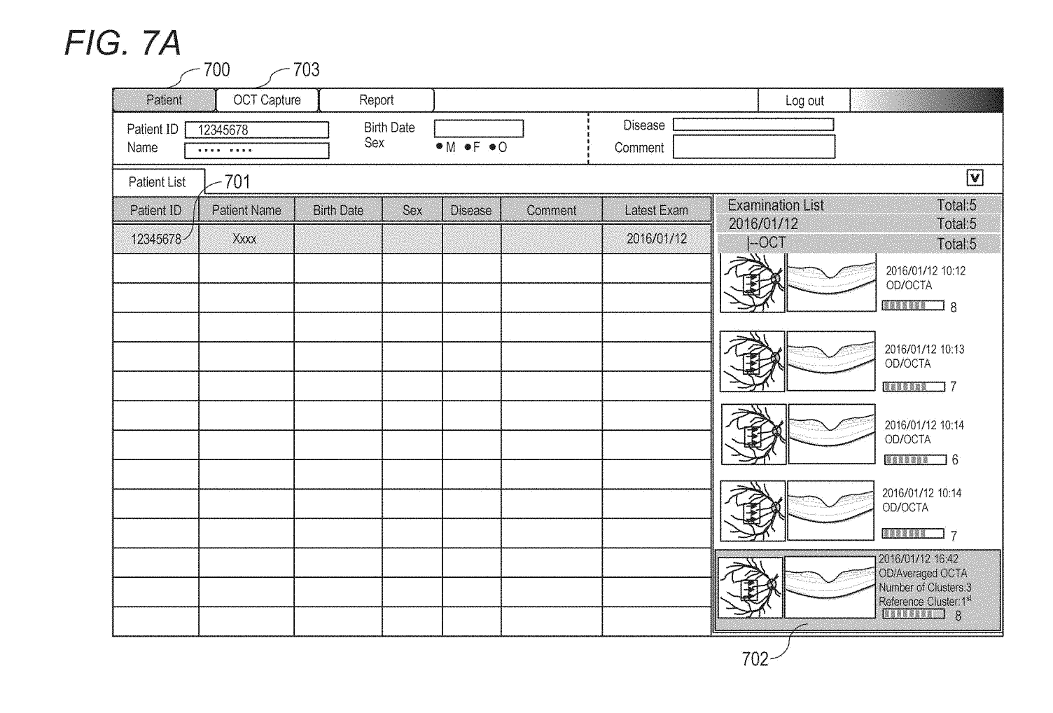

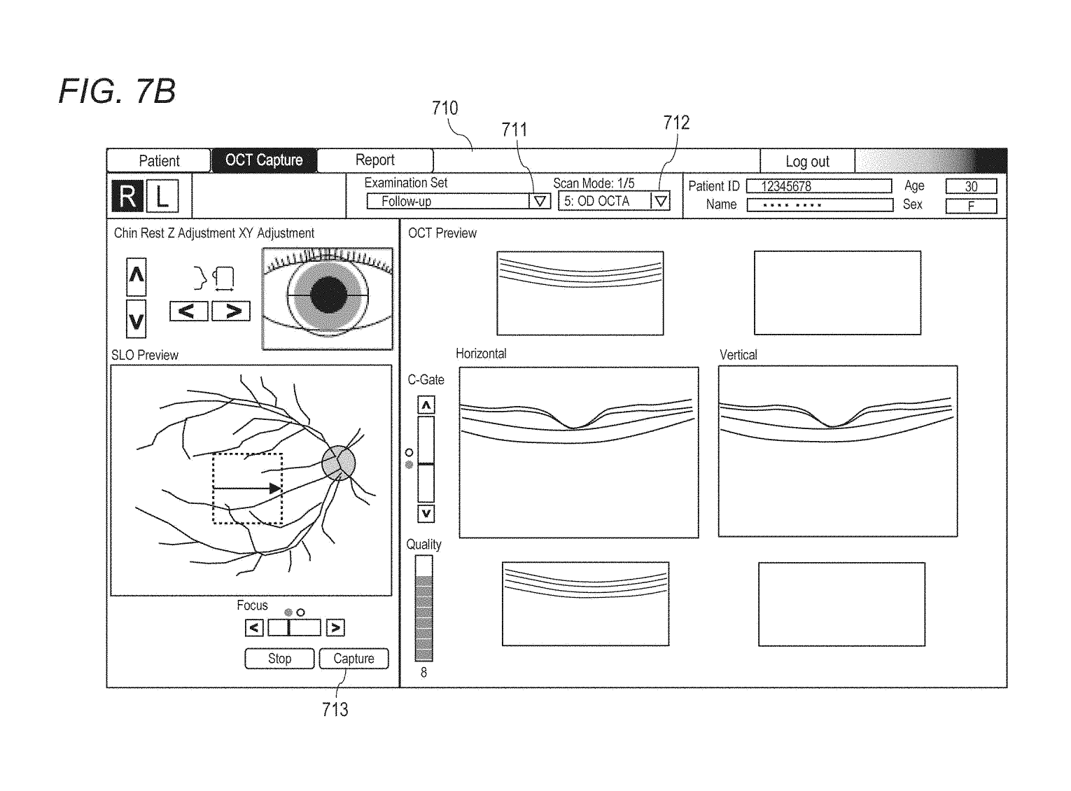

In the present embodiment, the operator selects an examinee 701 from a patient list (Patient List) by operating the input unit 103 on a patient screen 700 shown in FIG. 7A. Further, the operator decides the reference examination by selecting a reference examination (Baseline) in a follow-up examination from an examination list (Examination List) of the examinee (702 in FIG. 7A). Regarding selection of an examination set and a scan mode, when the operator opens an image capturing screen (OCT Capture 703) while selecting the reference examination, the image processing device 101 selects a follow-up examination set and sets the scan mode to the same scan mode as that of the reference examination. Specifically, the image capturing control unit 101-03 acquires an image capturing condition (scan mode) associated with the reference examination. In the present embodiment, as shown in an image capturing screen 710 of FIG. 7B, “Follow-up” (711) is selected as the examination set and “OCTA” mode 712 is selected as the scan mode. Here, the examination set indicates an image capturing procedure (including the scan mode) set for each examination purpose and a predetermined display method of OCT image and OCTA image.

The image processing device sets an image capturing condition of the OCTA image to be specified to the tomographic image capturing device 100. As an image capturing condition regarding each OCTA image capturing, there are setting items as described below in (1) to (7). After setting these setting items to the same values as those of the reference examination, the OCTA image capturing (of the same image capturing condition) is repeatedly performed a predetermined times with appropriate breaks therebetween in S302. In the present embodiment, the OCTA image capturing where the number of B-scans per cluster is four is repeated three times.
(1) Scan pattern (Scan Pattern)
(2) Scan area size (Scan Size)
(3) Main scanning direction (Scanning Direction)
(4) Distance between scans (Distance between B-scans)
(5) Fixation lamp position (Fixation Position)
(6) Coherence gate position (C-Gate Orientation)
(7) The number of B-scans per cluster (B-scans per Cluster)

<Step 302>

The operator starts repetitive OCTA image capturing based on the image capturing condition specified in S301 by operating the input unit 103 and pressing an image capturing start (Capture) button 713 in the image capturing screen 710 shown in FIG. 7B.

The image capturing control unit 101-03 instructs the tomographic image capturing device 100 to perform the repetitive OCTA image capturing based on the setting indicated by the operator in S301, and the tomographic image capturing device 100 acquires a corresponding OCT tomographic image. The tomographic image capturing device 100 acquires an OCT tomographic image corresponding to the instruction from the image capturing control unit 101-03.

In this step, the tomographic image capturing device 100 also acquires an SLO image and performs tracking processing based on an SLO moving image. In the present embodiment, a reference SLO image used for the tracking processing in the repetitive OCTA image capturing is a reference SLO image set in first OCTA image capturing of a plurality of times of the OCTA image capturing, and a common reference SLO image is used in all the repetitive OCTA image capturing operations.

In the present embodiment, as cluster scanning, for example, B-scan image capturing is performed four times continuously inside a rectangular area of 3×3 mm with the fovea as a center of image capturing at each position in a vertical direction (sub-scanning direction) by defining a horizontal direction as a main scanning direction. A gap between cluster scanning lines adjacent to each other in the sub-scanning direction is 0.01 mm, and the OCT tomographic image is acquired by setting a coherence gate on a vitreous body side. In the present embodiment, one B-scan is composed of 300 A scans. The above numerical values are examples, and may be changed to other numerical values.

During the repetitive OCTA image capturing, for “selection of left or right eye” and “to perform or not to perform the tracking processing” in addition to the image capturing conditions set in S301, the same setting values as those of the reference examination are used (the setting values are not changed).

<Step 303>

The image acquiring unit 101-01 and the image processing unit 101-04 generate a motion contrast image based on the OCT tomographic image acquired in S302.

First, the tomographic image generating unit 101-11 generates a tomographic image of one cluster by performing, for example, wavenumber conversion, fast Fourier transform (FFT), and absolute value conversion (acquisition of amplitude) on the interfering signal acquired by the image acquiring unit 101-01.

Next, the positioning unit 101-41 performs positioning on tomographic images belonging to the same cluster and performs superimposition processing (additional average processing). The image feature acquiring unit 101-44 acquires layer boundary data from the superimposed tomographic images. In the present embodiment, a variable shape model is used as an acquisition method of the layer boundary. However, any known layer boundary acquisition method may be used. The acquisition processing of the layer boundary is not essential. For example, when a motion contrast image is generated only three-dimensionally and a two-dimensional motion contrast image projected in a depth direction is not generated, the acquisition processing of the layer boundary can be omitted. The motion contrast data generating unit 101-12 calculates a motion contrast between tomographic images adjacent to each other in the same cluster. In the present embodiment, as the motion contrast, a de-correlation value Mxy is obtained based on the following formula (1).

$$\begin{matrix}[ Expression 1 ] & \\ Mxy = 1 - 2 \times \frac{Axy \times Bxy}{{Axy}^{2} + {Bxy}^{2}} & ( 1 )\end{matrix}$$

Here, Axy indicates an amplitude (of complex number data after FFT processing) at a position (x, y) of tomographic image data A, and Bxy indicates an amplitude at the same position (x, y) of tomographic image data B. 0≤Mxy≤1 is established, and the larger a difference between both amplitude values, the closer the value of Mxy to 1. The motion contrast data generating unit 101-12 performs de-correlation calculation processing as shown in the formula (1) between arbitrary tomographic images temporally adjacent to each other (belonging to the same cluster). The motion contrast data generating unit 101-12 generates an image, which has an average value of obtained motion contrast values, the number of which is (the number of tomographic images per cluster−1), as a pixel value, as a final motion contrast image.

Here, the motion contrast is calculated based on the amplitude of the complex number data after RFT processing. However, the calculation method of the motion contrast is not limited to the above method. For example, the motion contrast data generating unit 101-12 may calculate the motion contrast based on phase information of the complex number data, or may calculate the motion contrast based on information of both the amplitude and the phase. Alternatively, the motion contrast data generating unit 101-12 may calculate the motion contrast based on the real part and/or the imaginary part of the complex number data.

In the present embodiment, the de-correlation value is calculated as the motion contrast. However, the calculation method of the motion contrast is not limited to this. For example, the motion contrast may be calculated based on a difference between two values, or the motion contrast may be calculated based on a ratio between two values.

Further, in the above description, the final motion contrast image is obtained by obtaining an average value of a plurality of acquired de-correlation values. However, the present disclosure is not limited to this. For example, an image having the median value or the highest value of the plurality of acquired de-correlation values as a pixel value may be generated as the final motion contrast image.

<Step 304>

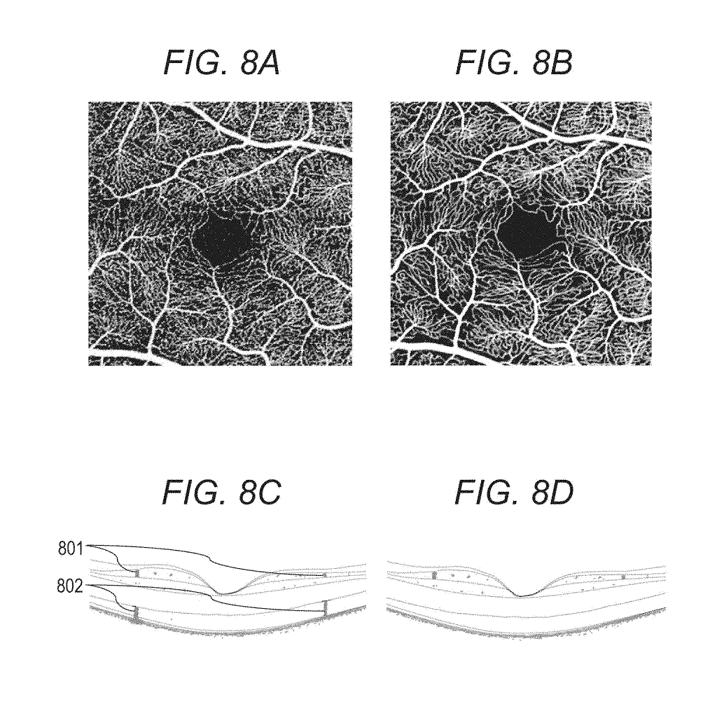

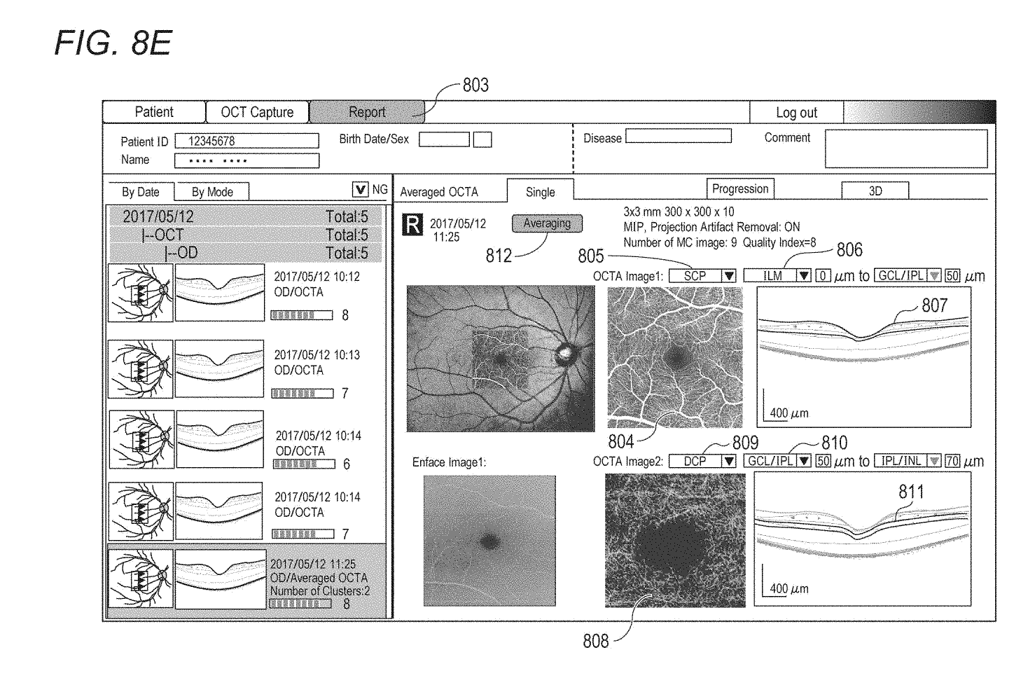

The image processing unit 101-04 three-dimensionally positions motion contrast image group obtained through the repetitive OCTA image capturing and additionally averages the motion contrast images. The image processing unit 101-04 generates a high-contrast synthesized motion contrast image as shown in FIG. 8B by additionally averaging a plurality of motion contrast images obtained from a plurality of clusters. FIG. 8A shows a motion contrast image obtained from one cluster for comparison with FIG. 8B. Synthesizing processing is not limited to simple additional average processing. For example, a value obtained by arbitrarily weighting luminance values of each motion contrast image and thereafter averaging the luminance values may be used, or an arbitrary statistical value such as a median value may be calculated. A case where the positioning processing is performed in a state of En Face image, that is, a case where the positioning processing is two-dimensionally performed, is also included in the present disclosure.

It may be configured so that the synthesizing unit 101-42 determines whether or not motion contrast images unsuitable for the synthesizing processing are included and then performs the synthesizing processing after removing the motion contrast images determined to be unsuitable. For example, when an evaluation value (for example, an average value of de-correlation values and fSNR) on a motion contrast image is outside a predetermined range, the synthesizing unit 101-42 may determine that the motion contrast image is unsuitable for the synthesizing processing.

In the present embodiment, the synthesizing unit 101-42 three-dimensionally synthesizes a motion contrast image, and then the correction unit 101-43 performs processing for three-dimensionally suppressing projection artifact generated in the motion contrast image.

Here, the projection artifact is a phenomenon Where a motion contrast in a retinal surface blood vessel is reflected on a deep layer side (retinal deep layer, retinal outer layer, and choroid) and a high de-correlation value is generated in an area on the deep layer side where there is actually no blood vessel. FIG. 8C shows an example where three-dimensional motion contrast data is superimposed on a three-dimensional OCT tomographic image. An area 802 having a high de-correlation value is generated on a deep layer side (photoreceptor cell layer) of an area 801 having a high de-correlation value corresponding to a retinal surface blood vessel area. Even though there is no blood vessel in the photoreceptor cell layer, blinking of blood vessel shadows generated in the retinal surface is reflected into the photoreceptor cell layer and a luminance value of the photoreceptor cell layer varies. Thereby, an artifact 809 occurs.

The correction unit 101-43 performs processing that suppresses a projection artifact 802 generated on a three-dimensional synthesized motion contrast image. Although any known projection artifact suppression method may be used, Step-down Exponential Filtering is used in the present embodiment. In the Step-down Exponential Filtering, processing shown by the formula (2) is performed on each A scan data on a three-dimensional motion contrast image, and thereby the projection artifact is suppressed.

$$\begin{matrix}[ Expression 2 ] & \\ {D}_{E} = ( x , y , z ) = D ( x , y , z ) \cdot {e}^{\frac{ {Σ }_{i = 1}^{z - 1} {D}_{E} ( x , y , i )}{γ}} & ( 2 )\end{matrix}$$

Here, γ represents an attenuation coefficient having a negative value, D(x, y, z) represents a de-correlation value before projection artifact suppression processing, and D_{E}(x, y, z) represents a de-correlation value after the projection artifact suppression processing.

FIG. 8D shows an example where three-dimensional synthesized motion contrast data (gray) after the projection artifact suppression processing is superimposed on a tomographic image. It is known that the artifact seen on the photoreceptor cell layer before the projection artifact suppression processing (FIG. 8C) is removed by the projection artifact suppression processing.

Next, the projection unit 101-45 projects the motion contrast image in a depth range based on the position of the layer boundary acquired by the image feature acquiring unit 101-44 in 5303 and generates a front motion contrast image. While the motion contrast image may be projected in an arbitrary depth range, in the present embodiment, two types of two-dimensional synthesized motion contrast images are generated in depth ranges of the retinal surface and the retinal deep layer. The projection unit 101-45 can select either of maximum intensity projection (MIP) and average intensity projection (AIP). In the present embodiment, the maximum intensity projection is used.

Finally, the image processing device 101 stores an acquired image group (SLO image and tomographic image), image capturing condition data of the image group, a generated motion contrast image, and accompanying generation condition data into the external storage unit 102 in association with examination date and information identifying the examinee eye.

<Step 305>

The display control unit 101-05 causes the display unit 104 to display the tomographic image generated in S303, the motion contrast image synthesized in S304, and information regarding the image capturing condition and a synthesis condition.

FIG. 8E shows an example of a report screen 803. In the present embodiment, the SILO image, the tomographic image, the front motion contrast images in different depth ranges generated by synthesizing and projecting in S304, and a corresponding front OCT image are displayed.

A projection range of the front motion contrast image can be changed when the operator selects a projection range from a predetermined depth range set (805 and 809) displayed in a list box. In the example in FIG. 8E, the retinal surface is selected in the list box 805, and the retinal deep layer is selected in the list box 809. Reference numeral 804 denotes an En Face image of the retinal surface, and reference numeral 808 denotes an En Face image of the retinal deep layer. The projection range can be changed by changing a type and an offset position of the layer boundary that are used to specify the projection range from a user interface such as 806 and 810 and/or operating and moving the layer boundary data (807 and 811) superimposed on a tomographic image from the input unit 103.

Further, an image projection method and the presence or absence of the projection artifact suppression processing may be changed by selecting those from a user interface such as a context menu.

<Step 306>

The operator instructs start of OCTA measurement processing by using the input unit 103.

In the present embodiment, when double-clicking a motion contrast image in the report screen 803 in FIG. 8E, an OCTA measuring screen as shown in FIG. 9A appears. The motion contrast image is enlarged and displayed, and a type of the image projection method (the maximum intensity projection (MIP) or the average intensity projection (AIP)), a projection depth range, and whether or not to perform projection artifact removal processing are appropriately selected. Next, the operator selects a type of measurement and a target area by selecting appropriate items from a selection screen 905 displayed through a Map button group 902, a Sector button group 903 and a Measurement button 904 on the right side of FIG. 9A, and then the analysis unit 101-46 starts measurement processing. At a time point when the OCTA measuring screen is displayed, no measurement target area is set (a state where None is selected in both the Map button group 902 and the Sector button group 903 and the selection screen 905 is not displayed). The analysis unit 101-46 starts measurement processing.

In the present embodiment, as a type of the measurement processing, one of the following (i) to (iii) is selected from the Map button group 902 or the Sector button group 903.
(i) None (no measurement is performed)
(ii) VAD (blood vessel density calculated based on the areas occupied by blood vessels)
(iii) VLD (blood vessel density calculated based on a total sum of lengths of blood vessels)

In addition to the above, for example, Fractal Dimension that quantifies complexity of blood vessel structure and Vessel Diameter Index that represents distribution of blood vessel diameters (distribution of knobs and stenoses of blood vessels) may be selected. One of the following (i) to (iv) can be selected from the selection screen 905 that is displayed through the Measurement button 904.
(i) Measurement of distance between arbitrary two points
(ii) Measurement of the area of avascular area
(iii) VAD
(iv) VLD

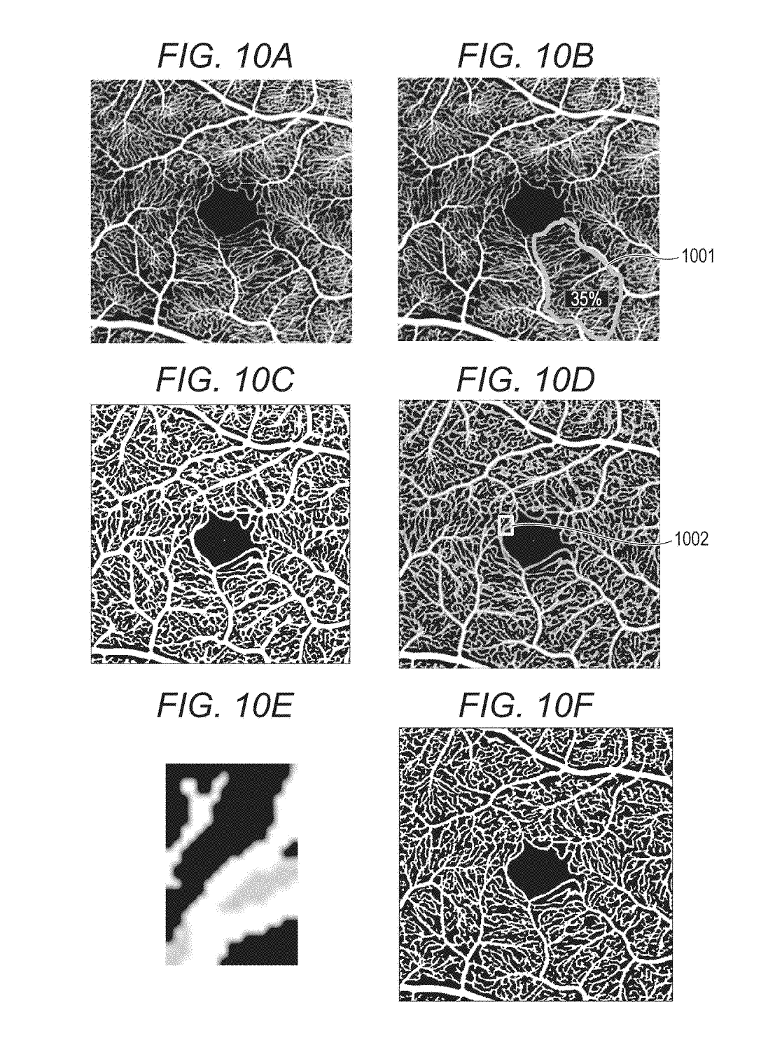

In the present embodiment, as a target area of the measurement processing, the entire image can be set by selecting a button other None from the Map button group 902, and a sector area (a smallest circle area and fan-shaped areas which have a fixation position as their center and which are defined by a plurality of concentric circles having different radii and a plurality of straight lines that pass through the fixation position and have different angles) can be set by selecting a button other than None from the Sector button group 903. Further, it is possible to set a measurement target area having an arbitrary shape by selecting a desired type of measurement from the selection screen 905 displayed through the Measurement button 904, specifying a boundary position (a gray line portion 1001 in FIG. 10B) having an arbitrary shape on the motion contrast image by using the input unit 103, and pressing an OK button. A numerical value shown in the area indicates a value measured in the area (VAD value in this case). When manually setting an area of interest, a circular control point indicating that the boundary position (the gray line portion 1001) is editable is superimposed on the specified boundary position. When the OK button is pressed, the circular control point disappears, only the gray line portion 1001 is displayed, and the boundary position becomes uneditable.

In the present embodiment, a case will be described where the VAD map (the type of measurement is VAD, the measurement target area is the entire image) and the VAD sector map (the type of measurement is VAD, the measurement target area is a sector area corresponding to ETDRS grid) are selected by selecting VAD from each of the Map button group 902 and the Sector button group 903.

Here, VAD is an abbreviation of Vessel Area Density and is a blood vessel density (unit: %) defined by a ratio of blood vessel area included in the measurement target. That is, VAD is an example of the blood vessel density regarding the area of the blood vessel area specified in the motion contrast image. VLD is an abbreviation of Vessel Length Density and is a blood vessel density (unit: mm⁻¹) defined by a total sum of lengths of blood vessels included per a unit area. That is, VLD is an example of the blood vessel density regarding the length of the blood vessel area specified in the motion contrast image. Further, VAD and VLD are an example of a parameter regarding the blood vessel area specified in the motion contrast image, Parameters regarding the blood vessel area include the area of the blood vessel area, a blood vessel length, a curvature of blood vessel, and the like.

Here, the blood vessel density is an index for quantifying a range of occluded blood vessels and the degree of coarseness and fineness of blood vessel network, and VAD is used most often for the blood vessel density. However, in VAD, a contribution of large blood vessel area to the measurement value is large, so that VLD is used (as an index sensitive to occlusion of capillary blood vessel) when it is desired to perform measurement by focusing attention on pathological condition of capillary blood vessels such as diabetic retinopathy. As the type of analysis, for example, there are parameters regarding an avascular area (Non Perfusion Area: NPA) specified in the motion contrast image in addition to the parameters regarding the blood vessel area. The parameters regarding the avascular area includes the area and the shape (the length and the degree of circularity) of the avascular area. In addition to the above, for example, Fractal Dimension that quantifies complexity of blood vessel structure and Vessel Diameter Index that represents distribution of blood vessel diameters (distribution of knobs and stenoses of blood vessels) may be measured.

A plurality of measurement target areas may be set for the same motion contrast image. Examples of the plurality of measurement target areas include at least two of the entire image, a sector area, and arbitrary shaped area, two or more depth ranges, and a combination of these. When different types of measurements are selected for the plurality of measurement target areas, measurement may be performed after interlockingly applying the type of analysis selected finally (for a specified measurement target area) to the other measurement target areas, and then a result of the measurement may be displayed. For example, in a state where the VAD map and the VAD selector map are selected, when an instruction to change to the VLD map is issued, the VLD sector map is automatically selected and a VLD measurement on the entire image and a VLD measurement on an ETDRS sector area are performed. By such an interlocking selection operation, it is possible to prevent a situation where different types of measurement values are superimposed for the same image and the operator is confused about displayed content.

None (no measurement is performed) of the types of measurement is independently selected in each measurement target area (When “None” is selected for a certain measurement target area, the selection of the “None” is not interlockingly applied to the other measurement target areas). The present disclosure is not limited to interlockingly applying the finally selected type of analysis to the all measurement target areas and performing measurement and display. For example, the finally selected type of analysis is interlockingly applied to a plurality of measurement target areas in an in-plane direction and is not interlockingly applied to a plurality of measurement target areas (the retinal surface and the retinal deep layer) in the depth direction. Alternatively, an operation opposite to the above-mentioned one is adaptively performed (the finally selected type of analysis is not interlockingly applied to a plurality of measurement target areas in the in-plane direction and is interlockingly applied to a plurality of measurement target areas in the depth direction). Then, measurement is performed and a corresponding measurement result may be displayed.

The VAD sector map and the VLD sector map can be moved based on an instruction from the input unit 103, and with this movement, values are recalculated by the measurement unit 101-463.

Next, the analysis unit 101-46 performs image enlargement and top-hat filter processing as preprocessing of the measurement processing. It is possible to reduce luminance variance of background component by applying the top-hat filter. In the present embodiment, an image is enlarged by using Bicubic interpolation so that a pixel size of the synthesized motion contrast image is about 3 μm, and the top-hat filter processing is performed by using a circular structural element.

<Step 307>

The analysis unit 101-46 performs identification processing of the blood vessel area. In the present embodiment, the enhancement unit 101-461 performs blood vessel enhancement processing based on a Hessian filter and edge selective sharpening. Next, the extraction unit 101-462 identifies the blood vessel area by performing binarization processing using two types of blood vessel enhanced images and performing shaping processing.

Details of the blood vessel area identification processing will be described in S510 to S560.

<Step 308>

The measurement unit 101-463 performs measurement of the blood vessel density on an image of a single examination based on information regarding a measurement target area specified by the operator. Subsequently, the display control unit 101-05 displays a measurement result on the display unit 104.

There are two types of indexes VAD and VLD as the blood vessel density. In the present embodiment, a procedure for calculating VAD will be described as an example. A procedure for calculating VLD will be described later.

When the operator inputs an instruction to modify the blood vessel area or the blood vessel center line data from the input unit 103, the analysis unit 101-46 modifies the blood vessel area or the blood vessel center line data based on positional information specified from the operator through the input unit 103 and recalculates measurement value.

When the measurement is performed without satisfying a predetermined condition in this step, the display control unit 101-05 outputs a message (warning display) indicating that the measurement should be performed in a state where the predetermined condition is satisfied to the display unit 104. Here, the predetermined condition is, for example, a condition where superimposition of OCTA images is performed.

Details of VAD measurement processing will be described in S810 to S830, and details of VLD measurement processing will be described in S840 to S870.

<Step 309>

The analysis unit 101-46 acquires an instruction indicating whether or not to modify the data of the blood vessel area and the blood vessel center line identified is S307 from outside. For example, the instruction is inputted by the operator through the input unit 103. When modification processing is instructed, the processing proceeds to S308, and when the modification processing is not instructed, the processing proceeds to S310.

<Step 310>

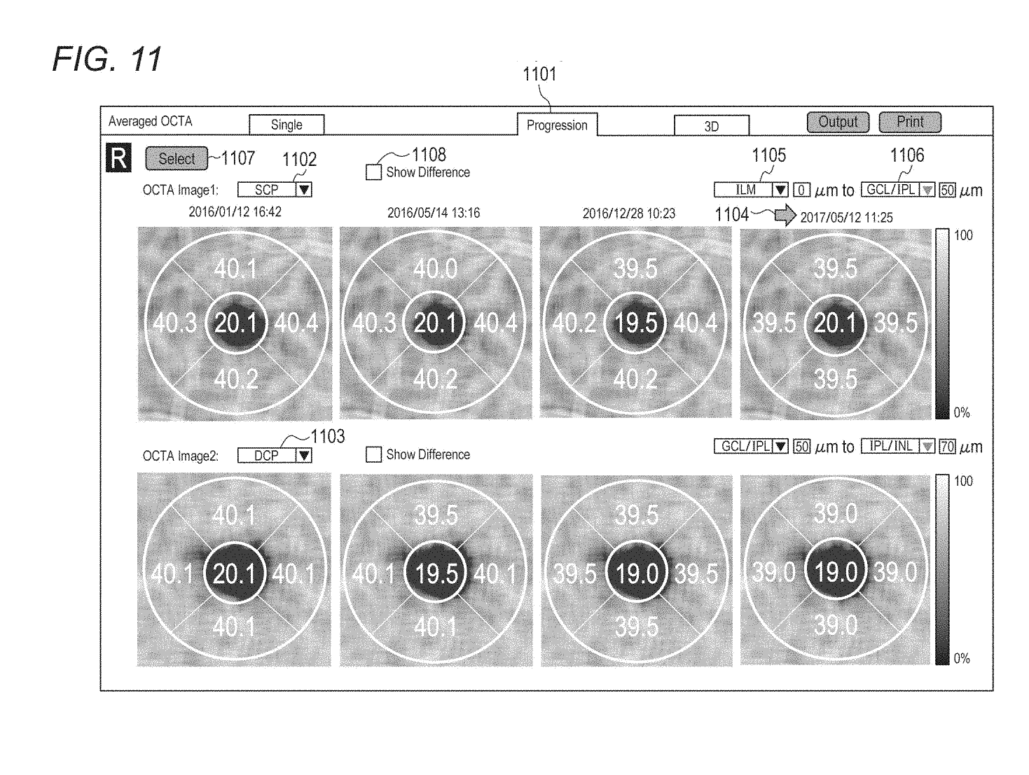

The comparison unit 101-464 performs temporal change measurement (Progression measurement) processing. FIG. 11 shows an example of a Progression measurement report. When specifying a Progression mode tab 1101, a screen of the report is displayed, and temporal change measurement processing based on the type of measurement and the measurement target area selected in S306 is started. In the present embodiment, as Progression measurement target images, the comparison unit 101-464 automatically selects four examinations in order of the examination date from the latest one. Furthermore, for example, an image of the oldest examination date and an image of the latest examination date, and images which are captured between the oldest and latest examination dates and which are captured at approximately equal intervals may be selected. The latest examination is, for example, an examination regarding the image capturing in S302.

Here, as selection conditions of the measurement target image, there are the following (i) and (ii) in descending order of priority. The selection conditions and the priority are not limited to the following example.
(i) The measurement target image is an image whose fixation position is the same.
(ii) The measurement target image is a motion contrast image where the number of tomographic images acquired in substantially the same position is large (for example, four or more) or a synthesized motion contrast image obtained by performing OCTA superimposition processing so as to be a motion contrast image equivalent to the above motion contrast image.

The comparison unit 101-464 preferentially selects images that satisfy the above selection conditions. For example, when an image of a second latest examination among latest five examinations whose fixation positions are the same is an OCTA image where superimposition is not performed and images of the other examinations are OCTA images where superimposition is performed, the comparison unit 101-464 selects the latest examination and the third to fifth latest examinations. Then, the display control unit 101-05 causes the display unit 104 to display the OCTA images of the selected examinations or information obtained from the selected OCTA images. In other words, the display control unit 101-05 does not display, in time series, motion contrast images that are not synthesized or information obtained from the motion contrast images that are not synthesized.

When the number of images that satisfy the selection conditions is less than four even though four images should be displayed, the display control unit 101-05 may cause the display unit 104 to display information indicating that there is no image to be displayed regarding images that does not satisfy the selection conditions or display the images with a display indicating that the images do not satisfy the selection conditions. For example, when there is only one OCTA image where superimposition is performed even though two images should be displayed, the OCTA image where superimposition is not performed may be displayed. In this case, information that distinguishes between the OCTA image where superimposition is performed and the OCTA image where superimposition is not performed may be displayed. For example, in FIG. 11, when the OCTA images are displayed in time series, information indicating a superimposed OCTA image (for example, a display of “AVG.”) is displayed for each image (for example, above the image) and “AVG.” is not displayed for OCTA images where superimposition is not performed.

When there are a plurality of images that satisfy the above (i) and/or (ii) as candidates to be displayed, the comparison unit 101-464 selects the latest image as an image to be displayed in time series. The merit of selecting the latest image is because the latest image is estimated to be an image which a doctor and the like consider preferable.

The measurement target image is not limited to this. For example, the measurement target image may be selected by selecting the Select button 1107 in FIG. 11 to display a selection screen and selecting the measurement target image from an image list displayed on the selection screen.

Next, the comparison unit 101-464 acquires an image of past examination and data regarding a measurement value corresponding to measurement content of a single examination performed in S309 from the external storage unit 102. The measurement value, which has been calculated in advance, may be acquired from the external storage unit 102, or the measurement value may be calculated after the image is acquired. Further, the positioning unit 101-41 performs positioning between the image of a single examination measured in S308 and a past examination image, and the comparison unit 101-464 generates measurement data (at least one of a measurement value, a measurement value map, a difference map, and a trend graph) regarding a common area. For the positioning, an OCTA image may be used or an SLO image may be used. The difference map is generated by specifying a “Show Difference” checkbox as shown by reference numeral 1108 in FIG. 11.

<Step 311>

The display control unit 101-05 displays a report regarding the Progression measurement performed in S310 on the display unit 104.

In the present embodiment, the VAD map and the VAD sector map measured in the retinal surface are superimposed in an upper part of the Progression measurement report shown in FIG. 11, and the VAD map and the VAD sector map measured in the retinal deep layer are superimposed in a lower part of the Progression measurement report. Thereby, it is possible to browse and grasp time-series changes of blood vessel disease in different depth positions. In the VAD measurement results juxtaposed and displayed in time series in FIG. 11, it is possible to browse and grasp a situation where an initial lesion occurs in the retinal deep layer and blood vessel blockages spread to the retinal surface or from the fovea to parafovea with the lapse of time. The display unit 104 may display the VLD map and the VLD sector map instead of the VAD map and the VAD sector map. Alternatively, the display unit 104 may display either maps or sector maps.

Instead of vertically juxtaposing and displaying information of the retinal surface and the retinal deep layer, information regarding VAD of the retinal surface and information regarding VLD of the retinal surface may be vertically juxtaposed and displayed. In other words, the display control unit 101-05 may cause the display unit 104 to juxtapose and display a plurality of pieces of information regarding a plurality of examination dates obtained from a combined image by different measurement methods.

in FIG. 11, the display control unit 101-05 may cause the display unit 104 to display a display indicating that displayed numerical values and the like are VAD. For example, the display control unit 101-05 may cause the display unit 104 to display a unit of VAD as a display indicating that the displayed numerical values and the like are VAD or display characters “VAD”. Also for VLD, the display control unit 101-05 may cause the display unit 104 to display a display indicating that displayed numerical values and the like are VLD. An image where the VAD sector map is superimposed may be a superimposed OCTA image instead of the VAD map. The display control unit 101-05 may cause the display unit 104 to display only the superimposed OCTA images in time series without displaying the VAD map and the VAD sector map. That is, the display control unit 101-05 corresponds to a display control unit that causes a display unit to display, in time series, a plurality of combined images regarding a plurality of examination dates or a plurality of pieces of information regarding a plurality of examination dates obtained from a plurality of combined images.

In the example shown in FIG. 11, the display control unit 101-05 causes the display unit 104 to display the VAD maps and the VAD sector maps of the retinal surface in an upper part of the display area and the VAD maps and the VAD sector maps of the retinal deep layer in a lower part of the display area. The retinal surface is an example of a first depth range, and the retinal deep layer is an example of a second depth range. In other words, the display control unit 101-05 causes the display unit to juxtapose and display, in time series, a plurality of pieces of information regarding the plurality of examination dates obtained from the combined images projected in the first depth range and a plurality of pieces of information regarding the plurality of examination dates obtained from the combined images projected in the second depth range different from the first depth range.

Regarding each measurement target image, the display control unit 101-05 may cause the display unit 104 to display information regarding the number of tomographic images in approximately the same position and an execution condition of OCTA superimposition processing and information regarding an evaluation value (image quality index) of the OCT tomographic image or the motion contrast image. In FIG. 11, a mark (“Averaged OCTA” in the upper left corner) indicating that the OCTA superimposition processing has been performed is displayed. An arrow 1104 displayed in the upper part of FIG. 11 is a mark indicating that this is the currently selected examination, and the reference examination (Baseline) is an examination (the leftmost images in FIG. 11) selected when Follow-up image capturing is performed. Of course, a mark indicating the reference examination may be displayed on the display unit 104. When “Show Difference” checkbox 1108 is selected in S310, a measurement value distribution (map or sector map) for a reference image is displayed on the reference image, and a differential measurement value map showing differences with the measurement value distribution calculated for the reference image is displayed in areas corresponding to other examination dates. As a measurement result, a trend graph (a graph of measurement values for images of each examination date obtained by the temporal change measurement) may be displayed on a report screen. A regression line (curved line) of the trend graph and/or a corresponding mathematical formula may be displayed on the report screen. It is possible to display VAD, VLD, and the size of avascular area as a trend graph. FAZ (Foveal Avascular Zone) may be displayed as the size of avascular area. The trend graph may be a graph showing values in an arbitrary area in a sector map, and an area displayed as the trend graph may be switchable by the input unit 103. The trend graph may be a graph that simultaneously displays graphs of each area of the sector map in a state where the graphs can be identified for each area. As the trend graph, VAD and VLD may be displayed on different coordinate systems, respectively, or on the same coordinate system. By doing so, a relationship between VAD and VLD can be easily grasped from the trend graph. Here, the trend graph may be displayed simultaneously with the map shown in FIG. 11 or a superimposed OCTA image, or may be displayed independently. Each of a plurality of measurement values included in the trend graph is a value regarding an image that satisfies a predetermined standard selected by the comparison unit 101-464, so that it is possible to accurately grasp temporal change.

In the present embodiment, images and measurement values of the retinal surface and the retinal deep layer as different depth ranges are displayed in time series. However, for example, images and measurement values in four depth ranges including the retinal surface, the retinal deep layer, the retinal outer layer, and the choroid may be displayed in time series. Further, the display unit 104 may display images and measurement values of arbitrary layers in time series.

Alternatively, the display unit 104 may juxtapose measurement values of different indexes and display them in time series. For example, the VAD maps may be juxtaposed and displayed in time series in an upper part, and the VLD maps (or shape values of an avascular area) may be juxtaposed and displayed in time series.

The projection depth range when the measurement values are juxtaposed and displayed in time series can be changed by using the user interfaces indicated by reference numerals 1102, 1103, 1105, and 1106 in FIG. 11 in the same manner as in the case of the user interfaces (805, 806, 809, and 810) in FIG. 8E described in S305. Further, similarly, the projection method (MIP/AIP) and the projection artifact suppression processing may be changed by a method such as, for example, selecting from a context menu. Furthermore, the type and the measurement target area of the Progression measurement can be changed by changing the type of measurement and items regarding the measurement target area into different values from a shortcut menu, and then the measurement can be performed again.

For example, items regarding the Map button group 902 in FIG. 9A and items regarding the Sector button group 903 in FIG. 9A are displayed on the shortcut menu, and one item is selected from each set of items (for example, “VLD Map” and “VLD Sector” are selected). In the same manner as in S306, when a plurality of measurement target areas are selected and the type of measurement for one area is changed, the same type of measurement is interlockingly applied to the other areas and the measurement is performed. An instruction that sets no measurement target area (an instruction where “None” is selected) is not interlockingly applied to the other measurement target areas.

The motion contrast image displayed on the display unit 104, and binary images, measurement values, and measurement maps regarding the blood vessel area and the blood vessel center line generated by the extraction unit 101-462 and the measurement unit 101-463 may be outputted to and stored in the external storage unit 102 as a file. To make comparative observation easy, image sizes and pixel sizes of the motion contrast images, and the binary images and the measurement maps regarding the blood vessel area and the blood vessel center line, which are to be outputted as a file, are desired to be the same.

Further, a warning message may be displayed when a result of measurement performed in a state where recommended conditions are not satisfied is displayed on a measurement report screen by the same method as in a case of measurement on a single examination (details will be described in S830). For example, a warning message described in S830 may be displayed on the display in FIG. 11. The recommended conditions are not limited to the conditions shown in S830. For example, “the number of tomographic images acquired in substantially the same position between selected temporal change measurement target images, or a synthesis condition of the motion contrast image, or a difference of image quality index value is less than a predetermined value” may be set as a recommended condition and a warning message may be displayed when the recommended condition is not satisfied.

<Step 312>

The image processing device 101 acquires an instruction whether or not to end a series of processing steps from S301 to S312 from outside. This instruction is inputted by the operator through the input unit 103. When receiving the instruction to end the processing, the image processing device 101 ends the processing. On the other hand, when the image processing device 101 receives an instruction to continue the processing, the image processing device 101 returns the processing to S302 and performs processing on the next examinee eye (or reprocessing on the same examinee eye).

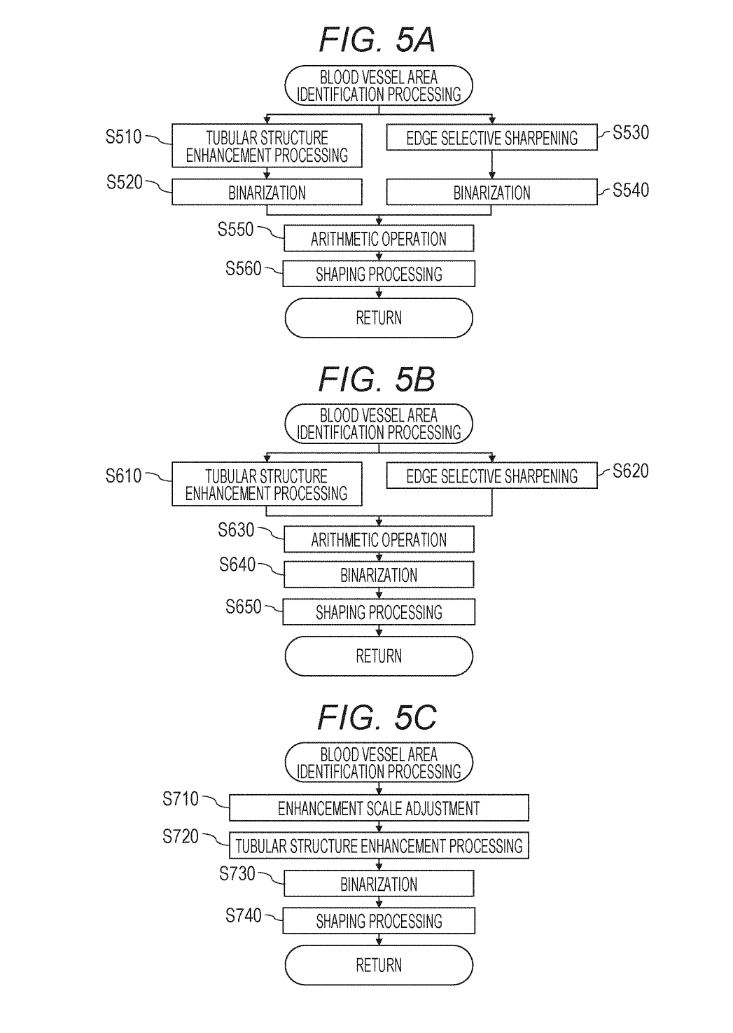

Further, details of the processing performed in S307 will be described with reference to a flowchart shown in FIG. 5A.

<Step 510>

The enhancement unit 101-461 performs blood vessel enhancement filter processing based on eigen values of Hessian matrix on the motion contrast image (OCTA image) on which the preprocessing of step S306 is performed. The enhancement filter is collectively called as a Hessian filter, and examples of the enhancement filter include Vesselness filter and Multi-scale line filter. In the present embodiment, the Multi-scale line filter is used. However, any known blood vessel enhancement filter may be used.

The Hessian filter smoothes an image in a size suited for a diameter of a blood vessel desired to be enhanced, and thereafter calculates a Hessian matrix having a secondary differential value of luminance value in each pixel of the smoothed image as an element, and enhances a local structure based on a magnitude relationship between the eigen values of the matrix. The Hessian matrix is a square matrix as given by a formula (3), and each element of the matrix is represented by a secondary differential value of a luminance value Is of an image obtained by smoothing a luminance value 1 of an image as shown by, for example, a formula (4), In the Hessian filter, when “one of eigen values (λ1, λ2) is close to 0 and the other is negative and has a large absolute value” in the Hessian matrix, it is assumed that the image has a linear structure, and the image is enhanced. This corresponds to an operation where pixels for which features of a blood vessel area on the motion contrast image, that is, “luminance change is small in a traveling direction and the luminance value significantly drops in a direction perpendicular to the traveling direction”, are established are assumed to have a linear structure and enhanced. In other words, the Hessian filter corresponds to an example of a linear structure enhancement filter.

The motion contrast image includes blood vessels having various diameters such as capillary blood vessels and arteriovenous vessels, so that a line-intensified image is generated by applying a Hessian matrix to an image smoothed by a Gaussian filter using a plurality of scales. For example, a scale corresponding to a blood vessel diameter of capillary blood vessel and a scale corresponding to a blood vessel diameter of a blood vessel near the optic disk may be used. Next, as shown by a formula (5), the line-intensified image is multiplied by a square of a smoothing parameter σ of the Gaussian filter as a correction coefficient and then an image is synthesized by maximum value calculation. Then, the combined image (hessian is outputted from the Hessian filter.

The Hessian filter is resistive to noise and has an advantage to improve continuity of blood vessels. On the other hand, practically, a maximum blood vessel diameter included in an image is often unknown in advance, so that there is a disadvantage that an enhanced blood vessel area tends to be thick in particular when the smoothing parameter is too large with respect to the maximum blood vessel diameter in the image.

Therefore, in the present embodiment, the blood vessel area is prevented from being too large by calculating with an image where a blood vessel area is enhanced by another blood vessel enhancement method described in S530.

$$\begin{matrix}[ Expression 3 ] & \\ H = [ \begin{matrix}{\partial }_{xx} {I}_{s} & {\partial }_{xy} {I}_{s} \\ {\partial }_{yx} {I}_{s} & {\partial }_{yy} {I}_{s}\end{matrix} ] & ( 3 ) \\ [ Expression 4 ] & \\ {\partial }_{xx} {I}_{s} = \frac{{\partial }^{2}}{\partial {x}^{2}} G ( x , y ; σ ) * I ( x , y ) & ( 4 ) \\ [ Expression 5 ] & \\ {I}_{hessian} ( x , y ) = {max}_{i} \{ {σ}_{i}^{2} {I}_{hessian} ( x , y ; {σ}_{i} ) \} & ( 5 )\end{matrix}$$

<Step 520>

The extraction unit 101-462 binarizes the blood vessel enhanced image which is formed through the Hessian filter and generated in S510 (hereinafter the blood vessel enhanced image is referred to as a Hessian enhanced image.

When binarizing the blood vessel enhanced image by using a luminance statistical value (average value, median value, or the like) of the Hessian enhanced image as a threshold value, the threshold value rises due to a high luminance area of a large blood vessel in, for example, an optic papilla portion, so that there is a case where extraction insufficiency of capillary blood vessel around papilla (RPC; Radial Peripapillary Capillary) occurs. Further, in an area such as the retinal deep layer where the avascular area tends to enlarge, the threshold value is too low, so that there is a case where the avascular area is falsely detected as a blood vessel.

Therefore, in the present embodiment, the threshold value is prevented from being too high in the optic papilla portion by using an average value of a Hessian enhanced image, which is synthesized by only enhanced images of low scale (partial scales lower than or equal to the threshold value among scales of a plurality of filters), as the threshold value. Further, false detection in the avascular area is suppressed by setting a lower limit value of the threshold value.

Here, the method of preventing the threshold value from being too high in the optic papilla portion is not limited to the method of binarizing the blood vessel enhanced image by using a statistical value of an enhanced image of low scale as the threshold value. For example, a similar effect can be expected when binarizing the blood vessel enhanced image by using an average value obtained in a case where when the luminance value on the Hessian enhanced image is higher than or equal to a predetermined value, the luminance value is assumed to be the predetermined value, as the threshold value. Alternatively, the blood vessel enhanced image may be binarized by using a robust estimator such as, for example, an M-estimator as the threshold value.

In the present embodiment, the synthesized motion contrast image is enhanced by the Hessian filter, so that the continuity of the binarized blood vessel area is further improved as compared with a case where a single motion contrast image is enhanced by the Hessian filter.

<Step 530>

The enhancement unit 101-461 performs edge selective sharpening processing on the synthesized motion contrast image which is generated in S306 and on which the top-hat filter has been applied.

Here, the edge selective sharpening processing is to perform weighted sharpening processing after largely weighting an edge portion (a portion where luminance difference is large) in an image. In the present embodiment, the edge selective sharpening processing is performed by performing unsharp mask processing on the synthesized motion contrast image by using an image where a Sobel filter is applied as a weight.

When the sharpening processing is performed with a small filter size, an edge of a small blood vessel is enhanced, so that when an image is binarized, the blood vessel area can be more accurately identified (it is possible to prevent a phenomenon where the blood vessel area becomes thick). On the other hand, there is much noise in a motion contrast image where the number of tomographic images in the same image capturing position is small, so that there is a risk that the noise in a blood vessel is also enhanced. Therefore, the noise enhancement is suppressed by performing the edge selective sharpening.

<Step 540>

The extraction unit 101-462 binarizes a sharpened image which is generated in S530 and on which the edge selective sharpening processing is performed. While any known binarization method may be used, in the present embodiment, the binarization is performed by using a luminance statistical value (average value or median value) calculated in each local area on the sharpened image as the threshold value.

However, in a large blood vessel area in the optic papilla portion, the set threshold value is too high, so that many holes are made in the blood vessel area on the binary image. Therefore, the threshold value is prevented from being too high in particularly the optic papilla portion by setting an upper limit value of the threshold value.

In the same manner as in S520, when the ratio of the avascular area occupied in an image is high, a case occurs where the threshold value is too low and a part of the avascular area is falsely detected as a blood vessel. Therefore, false detection is suppressed by setting a lower limit value of the threshold value.

In the same manner as in S520, the synthesized motion contrast image is edge-selective-sharpened, so that a binarized noise-shaped falsely detected area can be more reduced than a case where a single motion contrast image is edge-selective-sharpened.

<Step 550>

When both the luminance value of the binary image of the Hessian enhanced image generated in S520 and the luminance value of the binary image of the edge-selective-sharpened image generated in S540 are greater than zero, the extraction unit 101-462 extracts (segmentalizes) the images as blood vessel candidate images. By this calculation processing, it is possible to acquire binary images in which an area where the blood vessel diameter is overestimated as shown in the Hessian enhanced image and a noise area as shown in the edge-selective-sharpened image are both suppressed, the boundary position of the blood vessel is accurate, and the continuity of the blood vessel is good.

Since both binary images are binary images based on the synthesized motion contrast image, a binarized noise-shaped falsely detected area is reduced as compared with a binary image based on the single motion contrast image, and in particular the continuity of the capillary blood vessel area is improved. Further, they are the synthesized motion contrast images, so that the image quality and the luminance level between examinations are stabilized, and extraction performance of blood vessel is easily stabilized between examinations.

<Step 560>

The extraction unit 101-462 performs opening processing (performs expansion processing after contraction processing) and closing processing (performs contraction processing after expansion processing) of a binary image as shaping processing of blood vessel area. The shaping processing is not limited to this. For example, a binary image is labeled and a small area removal may be performed based on the area of each label. The present processing is not essential processing.

The binary image of blood vessel area can be obtained by the above S510 to S560. The binary image is an image where different labels are attached to a blood vessel and a region other than blood vessel. The binary image can be said to be a result of segmentation.

The method of extracting a blood vessel from the motion contrast image including blood vessels with various diameters (a scale used to enhance a blood vessel is adaptively determined) is not limited to the method described in S510 to S560. For example, as shown in S610 to S650 in FIG. 5B, the blood vessel area may be identified by performing binarization (S640) by using the luminance statistical value (for example, an average value) for an image to which a calculation (S630) of multiplying the luminance value of the Hessian enhanced image and the luminance value of the blood vessel enhanced image obtained by edge selective sharpening is applied as a threshold value. An upper limit value and a lower limit value can be set to the threshold value. S610 and S620 are the same processing as S510 and S530, and S650 is the same processing as S560.

Alternatively, as shown by S710 to S740 in FIG. 5C, the blood vessel may be enhanced by adaptively changing a range of the smoothing parameter a used when applying the Hessian filter depending on the fixation position and the depth range of the image (S710), applying the Hessian filter (S720), and performing binarization (S730). S740 is the same processing as S560. A scale can be set according to an image capturing region such as, for example, σ=1 to 10 in an optic papilla retinal surface, σ=1 to 8 in a macular area retinal surface, and σ=1 to 6 in a macular area retinal deep layer.

The binarization processing is not limited to threshold processing, but any known segmentation method may be used.

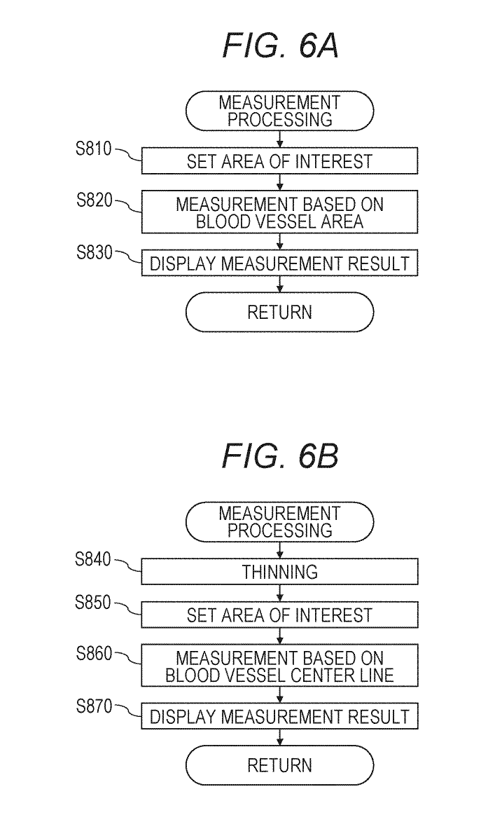

Further, details of processing performed in S308 will be described with reference to the flowchart shown in FIG. 6A.

<Step 810>

The operator sets an area of interest in the measurement processing through the input unit 103. In the present embodiment, in S306, as measurement content (type of measurement and measurement target area), a VAD map (the type of measurement is VAD and the measurement target area is the entire image) and a VAD sector map (the type of measurement is VAD and the measurement target area is a sector area corresponding to ETDRS grid) are selected. Therefore, as areas of interest, (i) the entire image and (ii) sector areas with the fixation lamp position as its center (areas obtained by dividing an annular area defined by an inner circle with a diameter of 1 mm and an outer circle with a diameter of 3 mm into four fan shapes Superior, Inferior, Nasal, and Temporal, and an area inside the inner circle) are set.

<Step 820>

The measurement unit 101-463 performs the measurement processing based on the binary image of the blood vessel area obtained in S307. In the present embodiment, a ratio of non-zero pixels (white pixels) in a neighboring area around a pixel is calculated at each pixel position of the binary image as the blood vessel density (VAD) at the pixel. Further, an image (VAD map) having values of the blood vessel density (VAD) calculated at each pixel is generated.

Then, a ratio of non-zero pixels (white pixels) in each sector area (set in S810) on the binary image is calculated as the blood vessel density (VAD) in the sector. Further, a map (VAD sector map) having values of the blood vessel density (VAD) calculated in each sector area is generated.

<Step 830>

The display control unit 101-05 displays the VAD map and the VAD sector map generated in S820 as measurement results on the display unit 104 In the present embodiment, in FIG. 9B, the VAD map of the retinal surface is displayed in a portion indicated by reference numeral 906 and the VAD map of the retinal deep layer is displayed in a portion indicated by reference numeral 908. Further, the VAD sector map of the retinal surface is superimposed on a portion indicated by reference numeral 907 and the VAD sector map of the retinal deep layer is superimposed on a portion indicated by reference numeral 909 in FIG. 9B.

In the present embodiment, the following conditions (i) to (iv) are set in FIG. 9B as recommended conditions of the measurement to be performed. At least one of the conditions (i) to (iv) needs to be used, and the recommended conditions are not limited to the following conditions.
(i) The measurement is performed on a motion contrast image where there are a predetermined number or more of tomographic images acquired in substantially the same position in a selected measurement target image. Alternatively, the measurement is performed on a synthesized motion contrast image where there are a predetermined number or more of tomographic images acquired in substantially the same position in the selected measurement target image. Alternatively, the measurement is performed on a motion contrast image whose image quality index value (Quality Index) is higher than or equal to a predetermined value.
(ii) The measurement is performed on a motion contrast image generated by the maximum intensity projection.
(iii) The projection artifact removal processing (PAR) has already been performed.
(iv) The measurement is performed on a motion contrast image generated in a projection depth range selected from projection depth ranges which include the retinal surface, the retinal deep layer, and a radial peripapillary capillary (RPC), respectively.

When the display control unit 101-05 displays a result of a measurement performed in a state where at least one of the conditions (i) to (iv) is not satisfied on the measurement report screen, the display control unit 101-05 assumes that the measurement has been performed in a condition where accurate measurement cannot be performed and displays warning.

For example, when displaying a result of a measurement performed in a state where the condition (i) is not satisfied, the display control unit 101-05 may cause the display unit 104 to display a warning message such as “Averaged OCTA is recommended in calculating VAD or VLD.” in, for example, lower right part in FIG. 9B (For example, lower right part in FIG. 12).

When displaying a result of a measurement performed in a state where the condition (ii) is not satisfied, the display control unit 101-05 may cause the display unit 104 to display a warning message such as “MIP is recommended in calculating VAD or VLD.” in, for example, lower right part in FIG. 9B.

Similarly, when displaying a result of a measurement performed in a state where the condition (iii) is not satisfied, the display control unit 101-05 may cause the display unit 104 to display a warning message such as “PAR is recommended in calculating VAD or VLD.” in, for example, lower right part in FIG. 9B.

Further, when displaying a result of a measurement performed in a state where the condition (iv) is not satisfied, the display control unit 101-05 causes the display unit 104 to display a warning message such as “Superficial Capillary, Deep Capillary, RPC can be analyzed in calculating VAD or VLD.”. By displaying the warning messages, it is informed to the public that a measurement result obtained by a measurement that does not satisfy the recommended measurement conditions has a risk to be a measurement result of low reliability, and by displaying a recommended measurement condition, a higher reliability measurement can be easily performed.

To avoid a situation where many warning messages occupy the report screen, it may be configured so that the recommended conditions described above are prioritized (for example, (i) is defined as a highest prioritized condition, (ii) is defined as a second highest prioritized condition, (iii) is defined as a third highest prioritized condition, and (iv) is defined as a fourth highest prioritized condition) and a warning regarding a measurement condition with highest priority among the unsatisfied measurement conditions is displayed. When displaying a plurality of measurement results as shown in FIG. 9B, warning messages may be displayed for respective measurements or only the highest priority warning message among the warning messages to be displayed may be displayed. Alternatively, in order to display warning messages regarding unsatisfied conditions without omission while making easy to understand conditions that largely affect reliability of the measurement result, the display control unit 101-05 may cause the display unit 104 to display warning messages regarding unsatisfied measurement conditions in a state where the priorities of the measurement conditions can be identified (by changing color and/or size). Examples of a case Where a plurality of measurement results are displayed include a case where the measurement results are respectively displayed in lower and upper parts of the report screen and a case where a plurality of measurement target areas are set for the same image and measurement results are displayed.

The warning message may be displayed in the same report screen or may be displayed as another screen. The warning message is not limited to a character string, but may be a still image or a moving image displayed on the display unit 104 or a voice to be outputted. A case where a report screen in Which the warning message is displayed is outputted as a file or a printed matter is also included in the present disclosure.

Further, a user interface may be included where the operator can select a warning message to be deleted from among the warning messages displayed on the display unit 104 by using the input unit 103 and/or the operator changes the priority of the warnings and/or specifies a warning message not to be displayed.

In the above description, a procedure for measuring VAD as the blood vessel density is described as an example. However, when generating the VLD map and the VLD sector map as measurement values, S840 to S870 shown in FIG. 6B are performed instead of S810 to S830 described above.

<Step 840>

The measurement unit 101-463 generates a binary image (hereinafter referred to as a skeleton image) whose line width is one pixel corresponding to a blood vessel center line by thinning the binary image of the blood vessel area generated in S307. An arbitrary thinning method or skeleton processing may be used. However, a thinning method of Hilditch is used as the thinning method in the present embodiment.

<Step 850>

The operator sets an area of interest similar to that in S810 through the input unit 103. In the present embodiment, the VLD map and the VLD sector map are calculated as measurement content (type of measurement and measurement target area). While VAD is selected in S810, VLD is selected in the present step, which is an only difference from S810. When the VLD map and the VLD sector map are not desired to be superimposed on the motion contrast image, the items of Map or Sector in FIG. 9A may be set to “None”.

<Step 860>

The measurement unit 101-463 performs measurement processing based on the skeleton image obtained in S840. In the present embodiment, a total sum of lengths of non-zero pixels (white pixels) per unit area [mm⁻¹] in a neighboring area around a pixel is calculated at each pixel position of the skeleton image as the blood vessel density (VLD) at the pixel. Further, an image (VLD map) having values of the blood vessel density (VLD) calculated at each pixel is generated.

Then, a total sum of lengths of non-zero pixels (white pixels) per unit area [mm⁻¹] in each sector area (set in S850) on the skeleton image is calculated as the blood vessel density (VLD) in the sector. Further, a map (VLD sector map) having values of the blood vessel density (VLD) calculated in each sector area is generated.

<Step 870>

The display control unit 101-05 displays the VLD map and the VLD sector map generated in S860 as measurement results in a portion indicated by reference numerals 906/907 or 908/909 in FIG. 9B.

In the same manner as in S830, when displaying a result of a measurement performed in a state where a condition suitable to a predetermined analysis is not satisfied on the measurement report screen, the display control unit 101-05 displays a warning message on the display unit 104.

In the present embodiment, a case where a measurement map is superimposed on the front motion contrast image has been described as a display method of blood vessel area identification and measurement results of a single examination. However, the display method is not limited to this. For example, a binary image and a skeleton image of an identified blood vessel area may be displayed in portions indicated by reference numerals 906 and 908 in FIG. 9B. Alternatively, a configuration where the motion contrast image is displayed in portions indicated by reference numerals 906 and 908 and the binary image or the skeleton image of the identified blood vessel area is superimposed on the motion contrast image after appropriately adjusting color or transparency parameter of the binary image or the skeleton image. The binary image is not limited to be displayed as a front image. For example, the binary image or the skeleton image of the identified blood vessel area may be superimposed on a B-scan tomographic image after appropriately adjusting color or transparency parameter of the binary image or the skeleton image.

When the operator inputs an instruction to modify the blood vessel area or the blood vessel center line data from the input unit 103 in S309, the blood vessel area or the blood vessel center line data are modified by a procedure as described below.

When a binary image including an excessive extraction area as shown in FIG. 10C is obtained with respect to the synthesized motion contrast image as shown in FIG. 10A, the analysis unit 101-46 deletes white pixels in a position specified by the operator through the input unit 103. Examples of a specification method of an addition/deletion position include a method of clicking the position while pressing a “d” key when specifying a deletion position and a method of clicking the position while pressing an “a” key when specifying an addition position. Alternatively, as shown in FIG. 10D, a binary image to be modified (a blood vessel area or a blood vessel center line) is superimposed on an image based on the motion contrast image after adjusting color and transparency parameter of the binary image, and thereby a state where an excessive extraction area or an insufficient extraction area can be easily distinguished is made. FIG. 10E shows an enlarged image of the inside of a rectangular area 1002 in FIG. 10D. A gray area indicates an excessive extraction area, and a white area indicates a de-correlation value of the original motion contrast image. It is possible to configure so that the operator specifies the excessive extraction area or the insufficient extraction area by using the input unit 103 and thereby a blood vessel or a blood vessel center line area on the binary image are accurately and efficiently modified. The modification processing of the binary image is not limited to the front image. For example, motion contrast data, binary data of blood vessel area, or a blood vessel center line area is superimposed on a B-scan tomographic image in an arbitrary slice position as shown by reference numeral 910 in FIG. 9A after adjusting color and transparency parameter of the motion contrast data, the binary data of blood vessel area, or the blood vessel center line area. After a state where an excessive extraction area or an insufficient extraction area can be easily distinguished is made in this way, the operator may specify and modify a three-dimensional position (x, y, z coordinates) of binary data to be modified (added/deleted) by the input unit 103.

Further, information indicating that the binary image (the binary image or the skeleton image of the blood vessel area) has been modified or information regarding a modified position is stored in the external storage unit 102 in association with the binary image, and the information indicating that the binary image has been modified or the information regarding the modified position may be displayed on the display unit 104 when displaying the blood vessel identification result and the measurement result on the display unit 104 in S870 or S311.

In the present embodiment, a case where the synthesizing unit 101-42 repeatedly generates the synthesized motion contrast image when completing the OCTA image capturing has been described. However, the generation procedure of the synthesized motion contrast image is not limited to this. For example, a synthesized motion contrast image generation instruction button 812 is arranged on the report screen 803 in FIG. 8E. The image processing device 101 may be configured so that the synthesizing unit 101-42 generates the synthesized motion contrast image when the operator explicitly presses the generation instruction button 812 after the OCTA image capturing is completed (may be on a date after the day on which the OCTA image capturing is performed). When the operator explicitly presses the synthesized motion contrast image generation instruction button 812 and generates the combined image, a synthesized motion contrast image 804, synthesis condition data, and items regarding the combined image on an examination image list are displayed on the report screen 803 as shown in FIG. 8E.

When the operator explicitly presses the generation instruction button 812, the display control unit 101-05 performs the following processing: The display control unit 101-05 displays a synthesis target image selection screen, and the synthesizing unit 101-42 generates the synthesized motion contrast image and causes the display unit 104 to display the synthesized motion contrast image when the operator operates the input unit 103 to specify a synthesis target image group and presses a permission button. A case where the synthesized motion contrast image that has been generated is selected and synthesized is also included in the present disclosure.

When the operator presses the synthesized motion contrast image generation instruction button 812, a two-dimensional combined image may be generated by synthesizing two-dimensional images obtained by projecting a three-dimensional motion contrast image, or a two-dimensional combined image may be generated by generating a three-dimensional combined image and then projecting the three-dimensional combined image.

According to the configuration described above, the image processing device 101 performs the blood vessel area identification processing and the blood vessel density measurement processing by using the front motion contrast images of the retinal surface and the retinal deep layer generated from an OCTA superimposed image acquired from the same examinee eye on different dates in substantially the same image capturing condition. The image processing device 101 juxtaposes and displays combined images and measurement values, which are obtained by the identification processing and the measurement processing, in time series in a plurality of depth ranges.

The OCTA superimposed image is used, so that it is possible to suppress influence of variation of signal intensity and image quality of the OCT tomographic images for each examination. As a result, according to the present disclosure, it is possible to support appropriate evaluation of temporal change regarding a fundus oculi blood vessel. Specifically, according to the present embodiment, it is possible to accurately identify and measure changes of blood vessel disease. Further, for example, it is also possible to accurately identify and measure changes of blood vessel diseases (blood vessel blockage, newborn blood vessel, lump on the blood vessel, and the like) while suppressing the influence of variation of signal intensity and image quality of the OCT tomographic images for each examination. Furthermore, it is possible to quantitatively grasp, for example, distribution of blood vessel disease by performing an analysis on one OCTA image.

### Second Embodiment

An image processing device according to the present embodiment is configured to three-dimensionally perform the blood vessel area identification processing and the measurement processing in the first embodiment and juxtapose and display obtained images and measurement data (measurement data of the blood vessel area and the blood vessel center line) in time series.

Specifically, motion artifact suppression processing is performed on a three-dimensional synthesized motion contrast image including a choroidal neovascular (CNV, Choroidal NeoVasucularization). Next, a blood vessel area including CNV is three-dimensionally identified by binarizing the image by applying three-dimensional morphologic filer and blood vessel enhancement filter. Further, a case of displaying, in time series, blood vessel densities calculated in the retinal surface and the retinal deep layer, and binary images and cubic content values of a choroidal neovascular area that are identified and measured in the retinal outer layer will be described.

The configuration and image processing flow of the image processing system 10 including the image processing device 101 according to the present embodiment are the same as those of the first embodiment, and therefore the description thereof will be omitted.

The image processing flow of the present embodiment other than S306 to S308 and S310 to S311 in FIG. 3 is the same that of the first embodiment, and therefore the description thereof will be omitted.

<Step 306>

The operator instructs start of OCTA measurement processing by using the input unit 103.

in the present embodiment, when double-clicking a motion contrast image in the report screen 803 in FIG. 8E, an OCTA measuring screen as shown in FIG. 9A appears. The motion contrast image is enlarged and displayed, and a type of the image projection method (the maximum intensity projection (MIP) or the average intensity projection (AIP)), a projection depth range, and whether or not to perform projection artifact removal processing are appropriately selected. Next, the operator selects a type of measurement and a target area by selecting appropriate items from a selection screen 905 displayed through a Map button group 902, a Sector button group 903 and a Measurement button 904 on the right side of FIG. 9A, and then the analysis unit 101-46 starts measurement processing.

As a type of the measurement processing, one of the following (i) to (iv) is selected from the Map button group or the Sector button group.
(i) None (no measurement is performed)
(ii) VAD (blood vessel density calculated based on the areas occupied by blood vessels)
(iii) VLD (blood vessel density calculated based on a total sum of lengths of blood vessels)
(iv) Volume (cubic content of blood vessel area)

The type of measurement to be selected is not limited to the above types, and any type of measurement may be performed.

For example, instead of (iv) Volume, a case where the area of a blood vessel area (for example, choroid capillary blood vessel) (which is obtained by identifying a blood vessel on a two-dimensional motion contrast image or projecting an identified three-dimensional blood vessel area in a predetermined depth range (for example, retinal outer layer) is calculated is also included in the present disclosure.

Further, one of the following (i) to (iv) is selected from the selection screen that is displayed through the Measurement button.
(i) Area measurement of avascular area
(ii) Blood vessel density (VAD)
(iii) Blood vessel density (VLD)
(iv) Cubic content of blood vessel area (Volume)

The type of measurement to be selected is not limited to the above types. For example, the area of a blood vessel area (for example, choroid capillary blood vessel) (which is obtained by identifying a blood vessel on a two-dimensional motion contrast image or projecting an identified three-dimensional blood vessel area in a predetermined depth range (for example, retinal outer layer) may be calculated.

The measurement performed by 3D image processing can be broadly classified into (1) to (3).
(1) Two-dimensional measurement on a blood vessel area or blood vessel center line data which are identified on an enhanced image which is three-dimensionally enhanced and two-dimensionally projected
(2) Two-dimensional measurement when projecting a blood vessel area or blood vessel center line data which are three-dimensionally enhanced and identified
(3) Three-dimensional measurement on a blood vessel area or blood vessel center line data which are three-dimensionally enhanced and identified

Examples of (1) and (2) include measuring the area of the avascular area, the blood vessel density, the area, a diameter, a length, and a curvature of the blood vessel area. The measurement content is the same as that on the front motion contrast image. However, measurement accuracy is improved because extraction performance of blood vessel is more improved than when enhancing, identifying, and measuring the front motion contrast image.

As examples of (3), there are the following examples.
(3-1) Cubic content measurement of a blood vessel
(3-2) Measurement on a cross-sectional image in an arbitrary direction or a curved cross-sectional image
(including a measurement of a diameter or a sectional area of a blood vessel)
(3-3) Measurement of a length and a curvature of a blood vessel

In the present embodiment, after three-dimensionally performing blood vessel enhancement processing and blood vessel area identification processing, VAD is measured on binary images projected in depth ranges of the retinal surface and the retinal deep layer, and a cubic content of a blood vessel area (choroidal neovascular area) is measured in a depth range of the retinal outer layer. A linear structure is enhanced by using the Hessian filter on a three-dimensional motion contrast image, so that it is possible to avoid unnecessarily enhancing a structure which is detected as a line in a two-dimensional motion contrast image but is not actually a line. As a result, it is possible to perform accurate segmentation (identification) of a blood vessel area.

In the same manner as in the first embodiment, it is possible to configure so that when one of the type of measurement selected from the Map button group and the type of measurement selected from the Sector button group is changed, the other is also changed interlockingly (to the same type of measurement).

Next, the analysis unit 101-46 performs image enlargement and top-hat filter processing as preprocessing of the measurement processing. In the present embodiment, three-dimensional Bicubic interpolation and three-dimensional top-hat filter processing are performed.

<Step 307>

The analysis unit 101-46 performs identification processing of the blood vessel area. In the present embodiment, the enhancement unit 101-461 performs blood vessel enhancement processing based on a three-dimensional Hessian filter d three-dimensional edge selective sharpening filter processing. Next, in the same manner as in the first embodiment, the extraction unit 101-462 identifies the blood vessel area by performing binarization processing using two types of blood vessel enhanced images and performing shaping processing.

Details of the blood vessel area identification processing will be described in S510 to S560.

<Step 308>

The measurement unit 101-463 performs measurement on an image of a single examination based on information regarding a measurement target area specified by the operator. Subsequently, the display control unit 101-05 displays a measurement result on the display unit 104.

In the same manner as in the first embodiment, when displaying a result of a measurement performed in a state where a condition suitable to a predetermined analysis is not satisfied on the measurement report screen, the display control unit 101-05 displays a warning message on the display unit 104.

When the operator inputs an instruction to modify the blood vessel area or the blood vessel center line data from the input unit 103, in the same manner as in the first embodiment, the analysis unit 101-46 modifies the blood vessel area or the blood vessel center line data based on positional information specified from the operator through the input unit 103 and recalculates measurement value.

The VAD measurement in the retinal surface and the retinal deep layer and a cubic content measurement of the choroidal neovascular in the retinal outer layer will be described in S810 to S830, and the VLD measurement in the retinal surface and the retinal deep layer and a total blood vessel length measurement of the choroidal neovascular in the retinal outer layer will be described in S840 to S870.

<Step 310>

The comparison unit 101-464 performs temporal change measurement (Progression measurement) processing by the same operation as that in the first embodiment.

<Step 311>

The display control unit 101-05 displays a report regarding the Progression measurement performed in S310 on the display unit 104.

In the present embodiment, the VAD map measured in the retinal surface is displayed in the uppermost part of the Progression measurement report, the VAD map measured in the retinal deep layer is displayed in a second part of the Progression measurement report, and (i) and (ii) of the choroidal neovascular area measured in the retinal outer layer are juxtaposed and displayed in time series in a third part of the Progression measurement report.
(i) Binary image (or a difference image between the binary image and a reference image)
(ii) Cubic content value or total blood vessel length (or a difference value between the binary image and the reference image)

The display is not limited to this. For example, blood vessel density (VAD or VLD) maps in the choroid may be juxtaposed and displayed in time series in a fourth part of the Progression measurement report.

Thereby, it is possible to browse and grasp time-series changes of three-dimensional disease of a fundus oculi blood vessel.

In the same manner as in the first embodiment, regarding each measurement target image, information of the number of tomographic images in approximately the same position, whether or not to perform the OCTA superimposition processing, an execution condition of the OCTA superimposition processing, and an evaluation value of the OCT tomographic image or the motion contrast image may be displayed on the display unit 104. Furthermore, the type and the measurement target area of the Progression measurement can be changed by changing the type of measurement and items regarding the measurement target area into different values from a shortcut menu, and then the measurement can be performed again. In the same manner as in the first embodiment, when a plurality of measurement target areas are selected and the type of measurement for one area is changed, the same type of measurement is interlockingly applied to the other areas and the measurement is performed. Further, when displaying a result of a measurement performed in a state where a predetermined condition is not satisfied on the measurement report screen, a warning message may be displayed by the same method as that in the first embodiment.

The present disclosure is not limited to a time series display of front images in different depth ranges and measurement value distributions for the front images, but it is possible to display, in time series, for example, images perpendicular to the front images and measurement value distributions for the images perpendicular to the front images, and volume-rendered three-dimensional images and measurement value distributions for the three-dimensional images.

Further, details of the processing performed in S307 will be described with reference to the flowchart shown in FIG. 5A.

<Step 510>

The enhancement unit 101-461 performs three-dimensional blood vessel enhancement filter processing based on eigen values of Hessian matrix on the motion contrast image on which the preprocessing of step 306 is performed. In the present embodiment, a three-dimensional Multi-scale line filter is used. However, any known blood vessel enhancement filter may be used.

In a three-dimensional Hessian filter, when “one of eigen values (λ1, λ2, λ3) is close to 0 and the others are negative and have a large absolute value” in a Hessian matrix (formula (6)) calculated in each pixel on a three-dimensional image, it is assumed that the image has a linear structure, and the image is enhanced.

$$\begin{matrix}[ Expression 6 ] & \\ H = ( \begin{matrix}{\partial }_{xx} {I}_{s} & {\partial }_{xy} {I}_{s} & {\partial }_{xz} {I}_{s} \\ {\partial }_{yx} {I}_{s} & {\partial }_{yy} {I}_{s} & {\partial }_{yz} {I}_{s} \\ {\partial }_{zx} {I}_{s} & {\partial }_{zy} {I}_{s} & {\partial }_{zz} {I}_{s}\end{matrix} ) & ( 6 )\end{matrix}$$

When the three-dimensional Hessian filter is used, properties of “luminance change is small in a blood vessel traveling direction and the luminance value significantly drops in two directions perpendicular to the blood vessel traveling direction” are obtained even for a blood vessel bending in the depth direction, so that there is an advantage to be able to satisfactorily enhance the blood vessel. Examples of blood vessels bending in the depth direction include the following three blood vessels.
Choroidal neovascular (CNV) penetrating into retina from choroid
Blood vessel in an optic papilla portion
Connecting portion between a retinal surface capillary blood vessel and a retinal deep layer capillary blood vessel

When a two-dimensional Hessian filter is applied to the above blood vessels on the front motion contrast image, in a two-dimensional plane, properties of “luminance change is small in a blood vessel traveling direction in the plane and the luminance value significantly drops in directions perpendicular to the blood vessel traveling direction” are not obtained, so that there is a problem that the blood vessels are not sufficiently enhanced and cannot be identified as blood vessel areas. When the three-dimensional Hessian filter is used, it is possible to satisfactorily enhance the above blood vessels, so that a blood vessel detection capability is improved.

<Step 520>

The extraction unit 101-462 binarizes the blood vessel enhanced image which is formed through the three-dimensional Hessian filter and generated in S510 (hereinafter the blood vessel enhanced image is referred to as a three-dimensional Hessian enhanced image.

The procedure of the binarization is similar to that of the first embodiment. However, the procedure is different from that of the first embodiment in that three-dimensional data is binarized. Further, the binarized image is an image formed by enhancing a synthesized motion contrast image by the Hessian filter, so that continuity of the binarized blood vessel area is improved as compared with a case where a single motion contrast image is enhanced by the Hessian filter.

<Step 530>

The enhancement unit 101-461 performs three-dimensional edge selective sharpening processing on the synthesized motion contrast image which is generated in S306 and on which the top-hat filter has been applied. In the present embodiment, the edge selective sharpening processing is performed by performing three-dimensional unsharp mask processing on the three-dimensional motion contrast image by using an image where a three-dimensional Sobel filter is applied as a weight.

<Step 540>

The extraction unit 101-462 binarizes a sharpened image which is generated in S530 and on which the edge selective sharpening processing is performed. While any known binarization method may be used, in the present embodiment, the binarization is performed by using a luminance statistical value (average value or median value) calculated in each three-dimensional local area on the three-dimensional sharpened image as the threshold value. In the same manner as in the first embodiment, extraction insufficiency in a blood vessel area and false extraction in an avascular area are suppressed by setting an upper limit value and a lower limit value of the threshold value.

In the same manner as in S520, the synthesized motion contrast image is edge-selective-sharpened, so that a noise-shaped falsely detected area is more reduced than a case where a single motion contrast image is edge-selective-sharpened.

<Step 550>

When both the luminance value of the binary image of the three-dimensional Hessian enhanced image generated in S520 and the luminance value of the binary image of the three-dimensional edge-selective-sharpened image generated in S540 are greater than zero, the extraction unit 101-462 extracts the images as blood vessel candidate images. By this calculation processing, it is possible to acquire binary images in which an area where the blood vessel diameter is overestimated as shown in the Hessian enhanced image and a noise area as shown in the edge-selective-sharpened image are both suppressed, the boundary position of the blood vessel is accurate, and the continuity of the blood vessel is good.

Since both binary images are binary images based on the synthesized motion contrast image, a binarized noise-shaped falsely detected area is reduced as compared with a binary image based on the single motion contrast image, and in particular the continuity of the capillary blood vessel area is improved. Further, they are the synthesized motion contrast images, so that the image quality and the luminance level between examinations are stabilized, and extraction performance of blood vessel is easily stabilized between examinations.

<Step 560>

The extraction unit 101-462 performs three-dimensional opening processing (performs expansion processing after contraction processing) and closing processing (performs contraction processing after expansion processing) as shaping processing of blood vessel area. The shaping processing is not limited to this, and when a binary image is labeled, a small area removal may be performed based on the area of each label.

In the same manner as in the first embodiment, the method of adaptively determining a scale used to enhance a blood vessel in the motion contrast image including blood vessels with various diameters is not limited to the method described in S510 to S560. For example, as shown in S610 to S650 in FIG. 5B, the blood vessel area may be identified by performing binarization by using the luminance statistical value (for example, an average value) for an image to which a calculation of multiplying the luminance value of the three-dimensional Hessian enhanced image and the luminance value of the blood vessel enhanced image obtained by three-dimensional edge selective sharpening is applied as a threshold value. An upper limit value and a lower limit value can be set to the threshold value.

Alternatively, as shown by S710 to S740 in FIG. 5C, the blood vessel may be enhanced by adaptively changing a parameter of a smoothing filter (the smoothing parameter σ of the Gaussian filter) when applying the Hessian filter based on three-dimensional positions of each pixel (or data of fixation position or depth range), applying the Hessian filter, and performing binarization.

The binarization processing is not limited to threshold processing, but any known segmentation method may be used.

Further, details of processing performed in S308 will be described with reference to the flowchart shown in FIG. 6A.

<Step 810>

The operator sets an area of interest in the measurement processing through the input unit 103.

In the present embodiment, the following (1) and (2) are calculated as measurement contents.
(1) VAD map and V AD sector map in the retinal surface and the retinal deep layer
(2) Cubic content of the choroidal neovascular in the retinal outer layer

Therefore, as areas of interest, the entire image and sector areas with the fixation lamp position as its center are selected in the retinal surface and the retinal deep layer. Further, in the retinal outer layer, a layer boundary corresponding to the retinal outer layer (a range surrounded by an OPL/ONL boundary and a position where a Bruch membrane boundary is moved toward the Bruch membrane boundary deep layer side by 20 μm) is unspecified.

<Step 820>

The measurement unit 101-463 performs the measurement processing based on the binary image of the blood vessel area obtained in S307. The measurement content (generation of the VAD map and the VAD sector map) in the retinal surface and the retinal deep layer is basically the same as that in the first embodiment. However, the measurement content is different from that of the first embodiment in that the measurement is performed after projecting three-dimensional blood vessel areas identified in the retinal surface and the retinal deep layer as front images. In the retinal outer layer, a cubic content of non-zero pixels (white pixels) in the area of interest corresponding to the retinal outer layer set in S810 is calculated.

<Step 830>

The display control unit 101-05 displays the VAD map and the VAD sector map in the retinal surface and the retinal deep layer, and a binary image of a blood vessel area in the retinal outer layer and a cubic content value of the blood vessel area, which are generated in S820 as measurement results, on the display unit 104.

When displaying a result of a measurement performed in a state where at least one of the recommended measurement conditions is not satisfied on the measurement report screen, a warning may be displayed (by assuming that the measurement has been performed in a condition where accurate measurement cannot be performed).

In the above description, a procedure of measuring a cubic content based on a specified three-dimensional blood vessel area is described as an example. However, when measuring a cubic content based on a three-dimensional blood vessel center line, S840 to S870 shown in FIG. 6B are performed instead of S810 to S830 described above.

<Step 840>

The measurement unit 101-463 generates a skeleton image whose line width is one pixel corresponding to a blood vessel center line by three-dimensionally thinning the binary image of the blood vessel area generated in S820.

<Step 850>

The operator sets an area of interest similar to that in S810 through the input unit 103. In the present embodiment, (1) and (2) are calculated as measurement contents.
(1) VLD map and VLD sector map in the retinal surface and the retinal deep layer
(2) total blood vessel length of the choroidal neovascular in the retinal outer layer

<Step 860>

The measurement unit 101-463 performs measurement processing based on the skeleton image obtained in S840. The measurement content (generation of the VLD map and the VLD sector map) in the retinal surface and the retinal deep layer is basically the same as that in the first embodiment. However, the measurement content is different from that of the first embodiment in that the measurement is performed after projecting three-dimensional skeletons identified in the retinal surface and the retinal deep layer as front images. Alternatively, the measurement may be performed after projecting a three-dimensional blood vessel area identified in S307 as a front image and then performing two-dimensional thinning processing. In the retinal outer layer, a total sum of lengths of non-zero pixels (white pixels) in the area of interest corresponding to the retinal outer layer set in S810 is calculated.

<Step 870 >

The display control unit 101-05 displays the VLD map and the VLD sector map in the retinal surface and the retinal deep layer, the skeleton image in the retinal outer layer, and a total sum of lengths of the skeleton on the display unit 104 generated in S860 as measurement results.

In the same manner as in S830, when displaying a result of a measurement performed in a state where a condition suitable to a predetermined analysis is not satisfied on the measurement report screen, a warning message is displayed.

In the present embodiment, a procedure for three-dimensionally extracting the choroidal neovascular and displaying cubic contents and total sums of blood vessel lengths in time series is described. However, the present disclosure is not limited to this. For example, by pressing the “Show Difference” checkbox in FIG. 11, a difference image and a difference value between the cubic content of the choroidal neovascular in the reference image and the cubic content of the choroidal neovascular in another image may be generated and displayed in time series.

Alternatively, by identifying an artery/vein area bending in the depth direction of the optic papilla portion in the same manner as the procedure described in the present embodiment, blood vessel shapes such as a blood vessel diameter, a blood vessel sectional area, and a curvature of blood vessel center line may be measured. Alternatively, a connection portion between a capillary blood vessel of the retinal surface and a capillary blood vessel of the retinal deep layer may be three-dimensionally extracted and highlighted, or the number of the connection portions may be counted.

According to the configuration described above, the image processing device 101 three-dimensionally identifies a blood vessel area by performing the motion artifact suppression processing on a three-dimensional synthesized motion contrast image and thereafter applying three-dimensional morphologic filer and blood vessel enhancement filter to the image and binarizing the image. Further, the image processing device 101 calculates the cubic content of the identified blood vessel area and displays binary images and cubic content values of the blood vessel area in time series.

Thereby, it is possible to accurately identify and measure changes of blood vessel disease while suppressing influence of variation of signal intensity and image quality of the OCT tomographic images for each examination.

### Third Embodiment

In the embodiments described above, a case where OCTA images obtained from each of a plurality of clusters are superimposed (and added and averaged) is mainly described. However, the present disclosure is not limited to this. For example, instead of the superimposed OCTA images, an OCTA image obtained by preparing nine or more tomographic images in one cluster may be used. By doing so, it is possible to obtain an OCTA image equivalent to an OCTA image in a case where the number of clusters is four and the number of tomographic images in each cluster is three.

### Fourth Embodiment

In the embodiments described above, two-dimensional OCTA images (motion contrast images) are displayed in time series in FIG. 11. However, three-dimensional OCTA images may be displayed in time series.

### Other Embodiments

Embodiment(s) of the present disclosure can also be realized by a computer of a system or apparatus that reads out and executes computer executable instructions one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2018-044559, filed Mar. 12, 2018, 2018-044560 filed Mar. 12, 2018, and 2018-044563 filed Mar. 12, 2018, which are hereby incorporated by reference herein in their entirety.

What is claimed is:

1. An image processing device comprising:
an acquiring unit configured to acquire combined images associated with examination dates, each of combined images being obtained by using motion contrast images of a portion of an eye portion; and
a display control unit configured to cause a display unit to display the combined images in time series and to cause the display unit to display a plurality of pieces of information regarding the examination dates in time series, the plurality of pieces of information being obtained by using the combined images.

2. The image processing device according to claim 1, wherein
the motion contrast images are three-dimensional motion contrast images obtained by controlling a measurement light so that measurement light scans the same position of the portion of the eye, and
the combined image is a combined image of the three-dimensional motion contrast images.

3. The image processing device according to claim 1, further comprising:
an analysis unit configured to perform an analysis on at least a partial area of the motion contrast image of the portion of the eye,
wherein when an image indicating a result of the analysis performed on the at least partial portion is an image obtained in a state where at least two conditions of a plurality of conditions suitable for the analysis are not satisfied, the display control unit causes the display unit to display information regarding the at least two conditions according to an order of priorities of the plurality of conditions.

4. The image processing device according to claim 3, wherein the display control unit causes the display unit to display a warning message regarding a higher priority condition of the at least two conditions as information regarding the at least two conditions along with an image indicating a result of analysis performed by using information indicating a type of analysis selected according to an instruction from an operator.

5. The image processing device according to claim 3, wherein the conditions include a condition that the motion contrast image where an analysis is performed on the at least partial portion is the combined image as a condition whose priority is higher than those of another condition of the plurality of conditions.

6. The image processing device according to claim 1, wherein the information is information regarding a measurement value calculated based on one of positions of a blood vessel area, an avascular area, and a blood vessel center line.

7. The image processing device according to claim 1, wherein the information is information of blood vessel density.

8. The image processing device according to claim 1, wherein the display control unit causes the display unit to display, in time series, a plurality of pieces of information regarding the examination dates obtained from the combined image projected in a first depth range and a plurality of pieces of information regarding the examination dates obtained from the combined image projected in a second depth range different from the first depth range, side by side.

9. The image processing device according to claim 1, wherein the display control unit causes the display unit to juxtapose and display a plurality of pieces of information regarding the examination dates obtained from the combined image by different measurement methods.

10. The image processing device according to claim 1, further comprising:
an analysis unit configured to perform an analysis on a first area in the motion contrast image of the portion of the eye by using information indicating a type of analysis selected according to an instruction from an operator,
wherein when display of an image showing a result obtained by analyzing a second area at least including an area smaller than the first area in the motion contrast image is selected according to an instruction from an operator, the display control unit causes the display unit to display the image showing the result obtained by analyzing the second area by using information indicating the type of selected analysis in a state where the image is superimposed on an image showing a result obtained by analyzing the first area.

11. The image processing device according to claim 1, further comprising:
an analysis unit configured to perform an analysis on at least one of a first area and a second area that at least includes an area smaller than the first area in the motion contrast image of the eye portion,
wherein the display control unit uses information indicating a type of analysis selected for one area of the first and the second areas according to an instruction from an operator and thereby causes the display unit to display an image showing a result where the one of the first and the second areas is analyzed, and when a type of analysis selected for the other area according to an instruction from an operator after selection for the one area is different from the type of analysis selected for the one area, in a display area of the image showing the result obtained by analyzing the one area, the display control unit performs control to change the display of the image showing the result obtained by analyzing the one area to a display of an image showing results obtained by analyzing the one area and the other area by using information indicating the type of analysis selected for the other area.

12. The image processing device according to claim 11, wherein when the type of analysis selected for the other area according to an instruction from an operator after selection for the one area is different from the type of analysis selected for the one area, the display control unit performs other control to change a display of the information indicating the type of analysis selected for the one area to a display of the information indicating the type of analysis selected for the other area.

13. The image processing device according to claim 11, wherein the display control unit also performs the control performed on one image of the combined images on the other images in a display area where the combined images are displayed in a time-sequential arrangement.

14. The image processing device according to claim 11, wherein the analysis unit applies information indicating a type of analysis selected for at least one of the first area and the second area in one image of the combined images to the other images according to an instruction from an operator.

15. The image processing device according to claim 11, wherein when the type of analysis selected for one area of the first and the second areas is changed to non-selecting according to an instruction from an operator, the type of analysis selected for the other area is not changed.

16. The image processing device according to claim 11, wherein when the type of analysis for the first area and the second area is non-selecting, the display control unit causes the motion contrast image to be displayed in the display area in a state where an image showing an analyzed result is not displayed in the display area.

17. The image processing device according to claim 11, wherein the selected type of analysis is one of types at least including a blood vessel density regarding an area of a blood vessel area identified in the motion contrast image and a blood vessel density regarding a length of the blood vessel area.

18. The image processing device according to claim 11, wherein
the motion contrast image is a motion contrast front image of the eye portion generated by using a three-dimensional motion contrast image of the eye portion and information regarding a depth range set according to an instruction from an operator,
the image showing a result of analysis is a two-dimensional image showing a result obtained by analyzing at least a partial area of the motion contrast front image,
the second area is a sector area,
the first area is an area larger than the sector area, and
the selected type of analysis is one of types at least including a parameter regarding a blood vessel area or an avascular area identified in the motion contrast image.

19. An image processing method comprising:
acquiring combined images associated with examination dates, each of combined images being obtained by using motion contrast images of a portion of an eye portion; and
causing a display unit to display the combined images, in time series, and to cause the display unit to display a plurality of pieces of information regarding the examination dates in time series, the plurality of pieces of information being obtained by using the combined images.

20. A non-transitory computer-readable storage medium storing instructions that when executed by one or more processors cause a computer to execute a method, the method comprising:
acquiring combined images associated with examination dates, each of combined images being obtained by using motion contrast images of a portion of an eye portion; and
causing a display unit to display the combined images, in time series, and to cause the display unit to display a plurality of pieces of information regarding the examination dates in time series, the plurality of pieces of information being obtained by using the combined images.
